(19) United States
(12) Patent Application Publication
(10) Pub. No.: US 2024/0058329 A1
(43) Pub. Date: Feb. 22, 2024

(54) OPHTHALMIC COMPOSITION OF RIFAMYCIN, AND USE THEREOF

(71) Applicants: BNC KOREA CO., LTD., Dalseogu,Daegu (KR); TEIKA PHARMACEUTICAL CO., LTD., Toyama-shi,Toyama (JP); AMD THERAPEUTICS LLC, Middletown, DE (US)

(72) Inventor: Hiroaki SERIZAWA, Palo Alto, CA (US)

(21) Appl. No.: 18/267,715

(22) PCT Filed: Dec. 21, 2021
(86) PCT No.: PCT/JP2021/047323

§ 371 Date: Jun. 15, 2023

(30) Foreign Application Priority Data
Dec. 21, 2020 (JP) 2020-211571

(51) Int. Cl. A61K 31/496; A61K 9/00; A61K 9/08; A61K 47/18; A61K 47/22; A61P 27/02

(52) U.S. Cl. CPC A61K 31/496; A61K 9/0048; A61K 9/08; A61K 47/186; A61K 47/22; A61P 27/02

(57) ABSTRACT

Provided is an ophthalmic composition comprising a rifamycin compound.

A topical eye drop composition, which comprises an aqueous solution formulation comprising an effective amount of a rifamycin compound selected from the group consisting of rifampicin, rifabutin, rifapentine, and rifaximin, or a pharmaceutically acceptable salt thereof, a pharmaceutically acceptable carrier, a buffer solution, and 0.00001% to 50% by weight of a nonionic surfactant.

### TECHNICAL FIELD

The present invention relates to a rifamycin ophthalmic composition.

In the present description, provided are: a pharmaceutically acceptable composition or a compositions preferable for topical administration to an eye, wherein the composition comprises a therapeutically effective amount of an antimicrobial agent, which, in a preferred embodiment, comprises a rifamycin derivative; a method for preparing these compositions, and a method for using these compositions in the treatment of various disorders.

### BACKGROUND ART

Loss of visual acuity is a common problem associated with aging, or with various diseases of eyes, such as macular degeneration, ocular histoplasmosis syndrome, myopia, diabetic retinopathy and inflammatory disease, all of which are caused by neovascularization in the cornea, retina or choroid.

Age-related macular degeneration (AMD) is a common eye disease, which usually affects elderly people and brings on a loss of vision in the center of the visual field (the macula) due to retinal damage. Although some peripheral vision remains, it is difficult or impossible to read or recognize faces. There are two major forms of macular degeneration, namely, atrophic (dry) and exudative (wet) forms. In dry (non-exudative) form, cellular debris called “drusen” accumulates between retina and choroid. In a more severe wet (exudative) form, blood vessels grow up from the choroid behind the retina. AMD is a leading cause of blindness among people older than 65 years and is caused by abnormal development of blood vessels behind retina. The advanced AMD population will increase by 11% and will reach 3.3 million due to the aging population. Intravitreal injection with anti-vascular endothelial growth factor (anti-VEGF) therapy has become the criterion standard for treatment of choroidal neovascular membranes (CNVs) associated with AMD. Treatment options for wet AMD include bevacizumab (Avastin, Genentech, San Francisco, CA), which is a full-length anti-VEGF antibody, ranibizumab (Lucentis, Genentech), which is an affinity-matured fragment, pegaptanib sodium (Macugen, OSI/Eyetech Inc.), and aflibercept (Eylea, Regeneron, Tarrytown, NY), and other anti-VEGF drugs. However, such intravitreal injection is a process that requires high precision, because it is performed with the help of a needle under local anesthesia. In this process, the needle must be inserted into the vitreous liquid that fills the cavity between the lens and retina, and thus, the operation must be carried out very carefully not to damage the retina. Accordingly, an easy treatment method for AMD is extremely desired. Therefore, additional treatment methods, and administration routes and methods for treating ocular disorders such as AMD are required.

On the other hand, since a rifamycin compound is extremely hardly soluble in water, it is difficult to produce formulations from the rifamycin compound.

Hence, various studies have been conducted to prepare an aqueous solution of the rifamycin compound. For example, the influence of the use of a surfactant, etc. has been reported (Patent Literature 1 and Non Patent Literature 1).

### CITATION LIST

### Patent Literature

Patent Literature 1: JP Patent Publication (Kohyo) No. 2017-525758 A

### Non Patent Literature

Non Patent Literature 1: SAEKO ANJO et al., Byouin Yakugaku (Hospital Pharmacy), Vol. 13, No. 5, pp. 288-292, 1987

### SUMMARY OF INVENTION

### Technical Problem

In order to enhance the solubility of a rifamycin compound in an aqueous vehicle, more detailed studies regarding conditions are required. It is particularly important to appropriately adjust the pH value. A change in the pH value of only about 1 greatly affects the solubility, and the concentration of a nonionic surfactant required is largely increased.

### Solution to Problem

The present inventor has conducted intensive studies directed towards solving the aforementioned problem. As a result, the aforementioned problem has been overcome by studying, in detail, mainly, a combination of the pH condition of a rifamycin compound and the concentration of a surfactant, thereby completing the present invention.

Specifically, the present invention relates to an ophthalmic pharmaceutical composition or formulation, comprising at least one rifamycin compound selected from the group consisting of, generally, rifampicin, rifabutin, and rifapentine, wherein the pharmaceutical composition or formulation is stable in an aqueous solution. In several aspects, the present invention is as follows.

[1] A topical eye drop composition, which comprises an aqueous solution formulation comprising an effective amount of a rifamycin compound selected from the group consisting of rifampicin, rifabutin, rifapentine, and rifaximin, or a pharmaceutically acceptable salt thereof, a buffer solution, and 0.00001% to 50% by weight of a nonionic surfactant, wherein
the pH of the aqueous solution formulation is pH 2 to 12, and
when the concentration (% by weight) of the nonionic surfactant and the concentration (% by weight) of the rifamycin compound satisfy the equation represented by the following formula (2) under any of the following conditions (i) to (iv), the rifamycin compound or a pharmaceutically acceptable salt thereof is stably dissolved in the aqueous solution formulation:

y_{Nis}=ax³+bx²+cx+d (2)
wherein y_{Nis} represents the concentration of the nonionic surfactant, and x represents the concentration of the rifamycin compound,
   (i) the aqueous solution formulation further comprises more than 0.00005% of an ionic surfactant, and when a buffer solution other than a boric acid buffer solution is used, the following holds in the formula (2):
(a, b, c, and d) (pH)=
(0, 0.106, 0.4548, 0.7308) (pH 3.0),
(0.0021, 0.0339, 2.5329, 0.3311) (pH 4.0),
(4.1739, −11.08, 22.977, −2.0291) (pH 5.0),
(3.9926, −8.618, 24.891, −2.2522) (pH 6.0),
(1.3898, −3.5786, 11.548, −0.9034) (pH 7.0) or
(−0.0364, 0.5336, 1.5747x, 0.2939) (pH 8.0),
   (ii) the aqueous solution formulation further comprises more than 0.00005% of an ionic surfactant, and when a boric acid buffer solution is used, the following holds in the formula (2):
(a, b, c, and d) (pH)=
(0, −0.1064, 3.0495, 0.0344) (pH 3.0),
(0.0185, −0.1476, 3.9692, −0.3971) (pH 4.0),
(−0.0732, 1.2435, 2.6415, −0.1436) (pH 5.0),
(0.1035, −0.1033, 3.9993, −0.3584) (pH 6.0),
(−0.0677, 0.5511, 2.7639, −0.1305) (pH 7.0) or
(−0.0032, 0.2011, 0.4421, 0.1908) (pH 8.0),
   (iii) the aqueous solution formulation further comprises 0.00005% or less of an ionic surfactant or does not comprise such an ionic surfactant, and when a buffer solution other than a boric acid buffer solution is used, the following holds in the formula (2):
(a, b, c, and d) (pH)=
(0, 0, 1.3762, 0.2364) (pH 3.0),
(0, 0, 2.901, 0.1891) (pH 4.0),
(0, 0, 17.396, −0.8064) (pH 5.0),
(0, 0, 20.916, −1.1493) (pH 6.0),
(0, 0, 11.115, −0.6384) (pH 7.0) or
(0, −0.8614, 4.3977, −0.1255) (pH 8.0), and
   (iv) the aqueous solution formulation further comprises 0.00005% or less of an ionic surfactant or does not comprise such an ionic surfactant, and when a boric acid buffer solution is used, the following holds in the formula (2):
(a, b, c, and d) (pH)=
(0, −0.2547, 4.0223, −0.497) (pH 3.0),
(0, 0, 3.0262, −0.1051) (pH 4.0),
(0, 0.7936, 2.607, 0.073) (pH 5.0),
(0, 0.2437, 4.7429, −0.7726) (pH 6.0),
(0, 0, 3.2942, −0.2059) (pH 7.0) or
(0, 0, 0.11, 0.6835, −0.1614) (pH 8.0).
   [2] A topical eye drop composition, which comprises an aqueous solution formulation comprising an effective amount of a rifamycin compound selected from the group consisting of rifampicin, rifabutin, rifapentine, and rifaximin, or a pharmaceutically acceptable salt thereof, a buffer solution, and optionally, an ionic surfactant and/or a nonionic surfactant, wherein
when the rifamycin compound, ionic surfactant, nonionic surfactant, and pH have the following relationship A1 or A2, the rifamycin compound or a pharmaceutically acceptable salt thereof is stably dissolved in the aqueous solution formulation:
   (A1) the aqueous solution formulation comprises 0% (not comprise) to 0.00005% by weight of an ionic surfactant and does not comprise a nonionic surfactant, and the concentration of the rifamycin compound is 0.002% to 10% by weight, and the pH of the aqueous solution formulation is pH 8.5 or more, or
   (A2) the aqueous solution formulation comprises more than 0.0005% by weight of an ionic surfactant, and the concentration of the nonionic surfactant is 0% (not added) to 15% by weight, and the concentration of the rifamycin compound is 0.0001% to 10% by weight, and the pH of the aqueous solution formulation is pH 8.5 or more.
      [3] The composition according to the above [1] or [2], further comprising a pharmaceutically acceptable carrier.
      [4] The composition according to the above [3], wherein the pharmaceutically acceptable carrier comprises a tonicity adjusting agent.
      [5] The composition according to the above [4], wherein the tonicity adjusting agent is saline, dextrose, glycerin, potassium chloride aqueous solution, buffer salts, propylene glycol, or mannitol.
      [6] The composition according to any one of the above [1] to [5], which is used for at least one selected from the group consisting of macular degeneration, diabetic retinopathy, chronic glaucoma, retinal detachment, sickle cell retinopathy, age-related macular degeneration (AMD), retinal ganglion cell injury, rubeosis iritis, inflammatory disease, chronic uveitis, neoplasm, Fuchs' heterochromic iridocyclitis, neovascular glaucoma, corneal neovascularization, choroidal neovascularization, retinal neovascularization, retinal angiomatous proliferation, glaucoma, glaucoma surgery, tissue adhesion, cicatrization, tissue fibrosis, and brain damage.
      [7] The composition according to any one of the above [1] to [6], further comprising one or more of an antibacterial agent, a pH buffer agent, an antioxidant, a preservative agent, or a combination thereof.
      [8] The composition according to any one of the above [1] to [7], wherein the rifamycin compound is rifampicin.
      [9] The composition according to any one of the above [1] and [3] to [7], wherein the concentration of the rifamycin compound or a pharmaceutically acceptable salt thereof is 0.1% to 10% (g/100 mL of the composition, weight/volume (w/v)).
      [10] A medicament for treatment of an ocular disease, disorder or condition, wherein the medicament comprises the composition according to any one of the above [1] to [9].
      [11] The medicament according to the above [10], wherein the ocular disease, disorder or condition is selected from age-related macular degeneration (AMD), ocular neovascularization, retinal ganglion cell injury, cicatrization, tissue fibrosis, and brain damage.

The above-described general description and detailed description are exemplification and explanation, and are intended to provide further explanation of the present invention as described in the scope of claims. Other objects, advantages, and novel features will be readily revealed to those skilled in the art from the following detailed description of the present invention.

### BRIEF DESCRIPTION OF THE DRAWINGS

FIG. 1-1 shows: histology sections of retinas treated with an AMD 101 topical eye drop formulation, an AMD 101 SC injection, and a control of only vehicle, in which oxygen-induced retinopathy was induced in the retinas; and a retina in which retinopathy was not induced.

FIG. 1A shows the 200× histological section of a retina treated with a control of only vehicle.

FIG. 1B shows the 400× histological section of a retina treated with a control of only vehicle.

FIG. 1C shows the 200× histological section of a retina treated with an AMD 101 topical eye drop formulation.

FIG. 1D shows the 400× histological section of a retina treated with an AMD 101 topical eye drop formulation.

FIG. 1-2 shows: histology sections of retinas treated with an AMD 101 topical eye drop formulation, an AMD 101 SC injection, and a control of only vehicle, in which oxygen-induced retinopathy was induced in the retinas; and a retina in which retinopathy was not induced.

FIG. 1E shows the 200× histological section of a retina treated with an AMD 101 SC injection.

FIG. 1F shows the 400× histological section of a retina treated with an AMD 101 SC injection.

FIG. 1G shows the 200× histological section of a retina, in which retinopathy was not induced.

FIG. 1H shows the 400× histological section of a retina, in which retinopathy was not induced.

FIG. 2 is a schematic diagram of a graph showing the solubility of the composition of the present invention.

FIG. 3 is a view showing the experimental results of the solubility of the composition of the present invention.

FIG. 4 is a view showing the experimental results of the solubility of the composition of the present invention, which are obtained regarding each rifampicin concentration, in which the used buffer solutions are divided into a phosphoric acid or citric acid buffer solution, and a boric acid buffer solution.

FIG. 5 is a view showing the relationship between the area under the curve (AUC) and the rifampicin concentration in the experimental results obtained in FIG. 4.

FIG. 6 is a view showing the relationship between the nonionic surfactant at each pH and the rifampicin concentration in the experimental results obtained in FIG. 4.

FIG. 7 is a view showing the experimental results of the solubility of the composition of the present invention, which are obtained in each rifampicin concentration, in which the used buffer solutions are divided into a phosphoric acid or citric acid buffer solution, and a boric acid buffer solution.

FIG. 8 is a view showing the relationship between the area under the curve (AUC) and the rifampicin concentration in the experimental results obtained in FIG. 7.

FIG. 9 is a view showing the relationship between the nonionic surfactant at each pH and the rifampicin concentration in the experimental results obtained in FIG. 7.

### DESCRIPTION OF EMBODIMENTS

In the present description, various embodiments will be described below. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). The description of the preferred embodiment as described herein, and as depicted in the drawings, is provided for illustrative purposes only.

The present invention relates to a topical eye drop composition, which comprises an aqueous solution formulation comprising an effective amount of a rifamycin compound selected from the group consisting of rifampicin, rifabutin, rifapentine, and rifaximin, or a pharmaceutically acceptable salt thereof, a pharmaceutically acceptable carrier, a buffer solution, and 0.00001% to 50% by weight of a nonionic surfactant, wherein
the pH of the aqueous solution formulation is pH 2 to 12, and
when the concentration (% by weight) of the nonionic surfactant and the concentration (% by weight) of the rifamycin compound satisfy the equation represented by the following formula (2) under any of the following conditions (i) to (iv), the rifamycin compound or a pharmaceutically acceptable salt thereof is stably dissolved in the aqueous solution formulation:

y_{Nis}=ax³+bx²+cx+d (2)
wherein y_{Nis} represents the concentration of the nonionic surfactant, and x represents the concentration of the rifamycin compound.

In the above formula (2), a, b, c, and d represent values that are changed depending on the concentration of an ionic surfactant or a nonionic surfactant in the aqueous solution formulation, the pH value, and the buffer solution used. The details thereof will be described later.

Moreover, the present invention relates to a topical eye drop composition, which comprises an aqueous solution formulation comprising an effective amount of a rifamycin compound selected from the group consisting of rifampicin, rifabutin, rifapentine, and rifaximin, or a pharmaceutically acceptable salt thereof, a buffer solution, and optionally, an ionic surfactant and/or a nonionic surfactant, wherein
when the rifamycin compound, ionic surfactant, nonionic surfactant, and pH have the following relationship A1 or A2, the rifamycin compound or a pharmaceutically acceptable salt thereof is stably dissolved in the aqueous solution formulation:
   (A1) the aqueous solution formulation comprises 0% (not comprise) to 0.00005% by weight of an ionic surfactant and does not comprise a nonionic surfactant, and the concentration of the rifamycin compound is 0.002% to 10% by weight, and the pH of the aqueous solution formulation is pH 8.5 or more, or
   (A2) the aqueous solution formulation comprises more than 0.0005% by weight of an ionic surfactant, and the concentration of the nonionic surfactant is 0% (not added) to 15% by weight, and the concentration of the rifamycin compound is 0.0001% to 10% by weight, and the pH of the aqueous solution formulation is pH 8.5 or more.

In the present invention, by combining a nonionic surfactant with a buffer solution, and also by specifically adjusting the concentration of the nonionic surfactant and the pH of the aqueous solution formulation to be between pH 1 and pH 12, the rifamycin compound was successfully dissolved in the aqueous solution formulation, without being deposited therein over a long period of time (for example, 2 hours, 5 hours, 10 hours, whole day and night, 1 day, 2 days, 3 days, 1 week, 2 weeks, 3 weeks, 1 month, 2 months, 3 months, or 6 months).

In addition, in the present invention, a combination of the concentration of a nonionic surfactant and the pH was studied in detail. As a result, conditions, under the rifamycin compound is stably dissolved in an aqueous vehicle even in the absence of a surfactant, were found.

Moreover, it was elucidated that addition of a nonionic surfactant to the tested composition is not necessary at all under basic conditions of pH 9 or more, or under acidic conditions of pH 3 or less. Furthermore, it was found that, in the case of using a boric acid buffer solution, the concentration of a nonionic surfactant that is required for solubilization is largely reduced, differing from the case of using other buffer solutions. These solubilization conditions are extremely varied, and thus, the solubilization conditions cannot be predicted from prior art documents at all. Further, such solubilization conditions are extremely important for preparing an ophthalmic composition, and it becomes possible to produce various compositions.

### Definitions

It must be noted that, as used in the present description and in the appended claims, the singular forms “a,” “an,” and “the” include plural referents, unless the context clearly dictates otherwise. Thus, for example, reference to “a solvent” (singular) includes a plurality of such solvents.

As used in the present description, the term “comprising” or “comprise” is intended to mean that the compositions and methods comprise the recited elements, but not excluding others. When the phrase “consisting essentially of,” when used to define compositions and methods, means excluding other elements having any essential significance to a combination for the stated purpose. Thus, a composition or process consisting essentially of the elements as defined herein would not exclude other materials or steps that do not materially affect the basic and novel characteristic(s) of the claimed invention. The phrase “consisting of” means excluding more than trace elements of other ingredients and substantial method steps. Embodiments defined by each of these transition terms are within the scope of the present invention.

Unless otherwise indicated, all numbers expressing quantities of ingredients, reaction conditions, and so on, which are used in the description and the claims are to be understood as being modified in all instances by the term “about.” Accordingly, unless otherwise indicated, the numerical parameters set forth in the following description and appended claims are approximate values. Each numerical parameter should, at least, be interpreted in light of the number of reported significant digits and by applying ordinary rounding techniques. The term “about,” when used before a numerical designation, e.g., temperature, time, amount, and concentration, including range, indicates approximate values, which may vary by (+) or (−) 20%, 15%, 10%, 5%, 4%, 3%, 2%, or 1%. However, even though the term “about” is not used, all numbers expressing quantities of ingredients, reaction conditions, and so on, do not exclude the above-described approximate values which may vary as experimental errors.

Combinations of substituents and variables are only those that result in the formation of stable compounds. The term “stable,” when used herein, refers to compounds, which possess stability sufficient to enable production, and which maintains the integrity of the compounds for a sufficient period of time, so that the compounds are useful for the purposes detailed herein (for example, 2 hours, 5 hours, 10 hours, whole day and night, 1 day, 2 days, 3 days, 1 week, 2 weeks, 3 weeks, 1 month, 2 months, 3 months, and 6 months).

As used in the present description, the term “hydrate” means a form of a compound, wherein water molecules are combined at a specific ratio as an essential part of the structure complex of the compound.

As used in the present description, the term “solvate” means a form of a compound, wherein solvent molecules are combined at a specific ratio as an essential part of the structure complex of the compound.

The term “pharmaceutically acceptable” or “pharmacologically acceptable,” when used herein, refers to a composition that is generally safe, non-toxic, and neither biologically nor otherwise undesirable and does not substantially produce adverse allergic or immunological reactions, when administered to a host (e.g., an animal or a human). Such a formulation includes any pharmaceutically acceptable dosage form.

The term “pharmaceutically acceptable salt” or “salt thereof” means a salt which is pharmaceutically acceptable, as defined above, and which has a desired pharmacological activity. Such salts include acid addition salts formed with organic and inorganic acids, such as hydrogen chloride, hydrogen bromide, hydrogen iodide, sulfuric acid, phosphoric acid, acetic acid, glycolic acid, maleic acid, malonic acid, oxalic acid, methanesulfonic acid, trifluoroacetic acid, fumaric acid, succinic acid, tartaric acid, citric acid, benzoic acid, and ascorbic acid. Base addition salts may be formed with organic and inorganic bases, such as sodium, ammonia, potassium, calcium, ethanolamine, diethanolamine, N-methylglucamine, and choline. Included are all pharmaceutically acceptable salts or compounds represented by the formulas used herein.

The term “pharmaceutically acceptable salt,” when used herein, refers to a pharmaceutically acceptable organic or inorganic acid or base salt of the compound, depending on the structure thereof. Representative examples of such a pharmaceutically acceptable salt may include alkali metal salts, alkaline-earth salts, ammonium salts, and water-soluble and water-insoluble salts, such as acetate, amsonate (4, 4-diaminostilbene-2, 2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, is othionate, lactate, lactobionate, laurate, malate, maleate, mandelate, mesylate, methylbromide, methylnitrate, methylsulfate, mucate, napsylate, nitrate, N-methylglucamine ammonium salt, 3 hydroxy 2 naphthoate, oleate, oxalate, palmitate, pamoate (1, 1-methene-bis-2-hydroxy-3-naphtho ate, einbonate), pantothenate, phosphate/diphosphate, picrate, polygalacturonate, propionate, p toluenesulfonate, salicylate, stearate, subacetate, succinate, sulfate, subsalicylate, suramate, tannate, tartrate, teoclate, tosylate, triethiodide, and valerate.

The terms “treat”, “treating” or “treatment”, when used herein, include alleviating, abating or ameliorating a disease or condition, or one or more symptoms thereof, preventing additional symptoms, ameliorating or preventing the underlying metabolic causes of symptoms, inhibiting the disease or condition, for example, arresting or suppressing the development of the disease or condition, relieving the disease or condition, causing regression of the disease or condition, relieving a condition caused by the disease or condition, or suppressing the symptoms of the disease or condition, and are also intended to include prophylaxis. The terms also include relieving the disease or conditions, for example, causing the regression of clinical symptoms. The terms further include achieving a therapeutic benefit and/or a prophylactic benefit. Such a therapeutic benefit is intended to mean eradication or amelioration of the underlying disorder being treated. Also, such a therapeutic benefit is achieved with the eradication or amelioration of one or more of the physiological symptoms associated with the underlying disorder such that an improvement is observed in the individual, notwithstanding that the individual is still be afflicted with the underlying disorder. For prophylactic benefit, the composition is administered to an individual at risk of developing a particular disease, or to an individual reporting one or more of the physiological symptoms of a disease, even though the diagnosis of this disease has not yet been made.

The terms “preventing” or “prevention” refer to a reduction in risk of acquiring a disease or disorder (i.e., causing at least one of the clinical symptoms of the disease not to develop in a subject that may be exposed to or predisposed to the disease but does not yet experience or display symptoms of the disease). The terms further include causing the clinical symptoms not to develop, for example, in a subject at risk of suffering from such a disease or disorder, thereby substantially averting onset of the disease or disorder.

The term “effective amount” refers to an amount that is effective for the treatment of a condition or disorder by intranasal administration of a compound or composition described herein. In some embodiments, an effective amount of any of the compositions or dosage forms, as described herein, is the amount used to treat a disorder mediated by hemoglobin, or a disorder that would benefit from tissue and/or cellular oxygenation of any of the compositions or dosage forms described herein to a subject in need thereof, or a disorder mediated by chronic or acute inflammation, or a disorder mediated by cicatrization or fibrosis.

The terms “carrier” and “vehicle,” when used herein, refer to relatively nontoxic chemical compounds or agents that facilitate the incorporation of a compound into cells, for example, ocular cells, or tissues. Carriers and vehicles useful herein include any given materials known in the present technical field, which are nontoxic and do not interact with other components of the formulation in a harmful manner. When used in the present description, the term “pharmaceutically acceptable carrier” includes any given excipients, and all solvents, dispersion media, coatings, wetting agents (e.g., sodium lauryl sulfate), isotonic and absorption delaying agents, disintegrants (e.g., potato starch or sodium starch glycolate), and tonicity adjusting agents.

Examples of the excipient may include viscosity imparting agents (cellulosic polymers, etc.), antioxidants (thiosulfate, sodium formaldehyde sulfoxylate, etc.), and tonicity adjusting agents (sodium chloride, etc.).

When used in the present description, the term “prodrug” means a compound that is metabolized or otherwise converted to an active or more active form with respect to at least one property, after administration. To produce a prodrug, a pharmaceutically active compound can be modified chemically to render it less active or inactive, but the chemical modification is such that an active form of the compound is generated by metabolic or other biological processes. A prodrug may have, relative to the drug, altered metabolic stability or transport characteristics, fewer side effects or lower toxicity. For example, see the reference Nogrady, 1985, Medicinal Chemistry A Biochemical Approach, Oxford University Press, New York, pages 388-392. Prodrugs can also be prepared using compounds that are not drugs.

The term “ophthalmically acceptable” with respect to the formulation, composition, or ingredient of the present description means having no persistent harmful effect on the treated eye or the functioning thereof, or on the general health of the subject being treated, exhibiting transient effects such as minor irritation or a “stinging” sensation.

The term “active agent” or “active ingredient” is used herein to refer to a chemical material or compound that induces a desired beneficial effect when administered to a patient. Also included are salts, derivatives and analogs of those compounds or classes of compounds specifically mentioned (e.g., rifamycin compounds) that also induce the desired effect. For example, the term “rifampicin” when used herein includes pharmaceutically acceptable salts thereof and derivatives thereof.

The terms “buffer solution” or “buffer agent” refer to materials, which, when added to a solution, cause the solution to resist changes in pH values.

The term “dilution” refers to dilution of the formulation of the present invention or those derived from the formulation of the present invention using, for example, an aqueous system comprised of physiologically balanced saline solution (PBS), such as phosphate buffered saline, or water, or other water soluble components, to the desired final concentration.

The term “subject,” when used herein, refers to an organism to be treated by the composition of the present invention. Such an organism includes animals (livestock animal species and wild animals), preferably mammals, including a human or a non-human mammal. The terms “patient” and “subject” may be used interchangeably.

The term “surfactant” refers to any given molecule having both a polar head group, which energetically prefers solvation by water, and a hydrophobic tail, which is not well solvated by water. The surfactant can be an ionic or a nonionic surfactant. The term “ionic surfactant” includes cationic, anionic, and zwitterionic surfactants. The term “cationic surfactant” refers to a surfactant with a cationic head group. The term “anionic surfactant” refers to a surfactant with an anionic head group.

The term “macular degeneration” refers to a variety of degenerative conditions characterized by central visual loss due to deterioration of the macula. One of these conditions is age related macular degeneration (AMD), which exists in both “dry” and “wet” forms.

The term “ocular neovascularization” refers to the abnormal development, proliferation, and/or growth of blood vessels on or in the eye, for example, on the retinal surface.

### Methods

In some embodiments provided in the present description, a method for treating visual disorders, such as age-related macular degeneration (AMD), ocular neovascularization, optic neuropathy, glaucoma, degeneration of optic nerves, and ophthalmoplegia, will be described. Any given ophthalmic formulations and/or compounds, as described above, are useful in the methods described in the present description.

In some embodiments, a medicament and a method for treating an ocular disease, disorder, injury or condition are provided, and this method comprises administering to a patient in need thereof, a medicament (an ophthalmic pharmaceutical composition) comprising an effective amount of a rifamycin compound selected from the group consisting of rifampicin, rifabutin, rifapentine, and rifaximin.

In some embodiments, the ocular disease, disorder, injury or condition is selected from the group consisting of macular degeneration, diabetic retinopathy, chronic glaucoma, retinal detachment, sickle cell retinopathy, age related macular degeneration (AMD), retinal ganglion cell injury, rubeosis iritis, inflammatory diseases, chronic uveitis, neoplasms, Fuchs' heterochromic iridocyclitis, neovascular glaucoma, corneal neovascularization, choroidal neovascularization, retinal neovascularization, retinal angiomatous proliferation, glaucoma, glaucoma surgery, tissue adhesion, cicatrization, fibrosis disorder, brain damage, and the like.

In some embodiments, a medicament and a method for treating ocular neovascularization are provided, and this method comprises administering to a patient in need thereof, a medicament (an ophthalmic pharmaceutical composition) comprising an effective amount of a rifamycin compound selected from the group consisting of rifampicin, rifabutin, rifapentine, and rifaximin. In some embodiments, the treatment reduces or reverses the loss of visual acuity secondary to neovascularization of cornea, iris, retina or choroid. In some embodiments, ocular neovascularization includes neovascularization generated as a result of a combination of vitrectomy and lensectomy, retinal ischemia, choroidal vascular insufficiency, choroidal thrombosis, carotid artery ischemia, neovascularization of the optic nerve, and neovascularization due to penetration of the eye or contusive ocular injury.

In some embodiments, a medicament and a method for treating age-related macular degeneration (AMD) are provided, and this method comprises administering to a patient in need thereof, a medicament (an ophthalmic pharmaceutical composition) comprising a therapeutically effective amount of a rifamycin compound selected from the group consisting of rifampicin, rifabutin, rifapentine, and rifaximin. In some embodiments, the method includes the treatment of a dry form of AMD. In other embodiments, the method includes the treatment of a wet form of AMD.

In some embodiments, a medicament and a method for treating retinal neovascularization are provided, and this method comprises administering to a patient in need thereof, a medicament (an ophthalmic pharmaceutical composition) comprising a therapeutically effective amount of a rifamycin compound selected from the group consisting of rifampicin, rifabutin, rifapentine, and rifaximin.

In some embodiments, a medicament and a method for protecting optic nerve cells are provided, and this method comprises administering to a patient in need thereof, a medicament (an ophthalmic pharmaceutical composition) comprising a therapeutically effective amount of a rifamycin compound selected from the group consisting of rifampicin, rifabutin, rifapentine, and rifaximin. In some embodiments, the optic nerve cells are retinal ganglion cells.

In some embodiments, a medicament and a method for inhibiting brain damage are provided, and this method comprises administering to a patient in need thereof, a medicament (an ophthalmic pharmaceutical composition) comprising a therapeutically effective amount of a rifamycin compound selected from the group consisting of rifampicin, rifabutin, rifapentine, and rifaximin.

In some embodiments of the present methods, the rifamycin compound is rifampicin. In some embodiments, the rifamycin compound is rifabutin. In some embodiments, the rifamycin compound is rifapentine. In some embodiments, the rifamycin compound is rifaximin. In some embodiments, the present ophthalmic composition further comprises at least one of a nonionic tonicity adjusting agent, a salt, a preservative agent, a buffer agent, a surfactant, an antioxidant, a solubilizing agent, and a stabilizer. In some embodiments, the composition is administered topically. In some embodiments, the present composition is in the form of eye drops, a gel, lotion, cream, or an ointment, or is incorporated into a conformer, an implant, a stent, or an ophthalmic spray drug delivery device.

The compound and medicament of the present invention may be used alone or in combination with other compounds. When administered together with another agent, co-administration can be carried out in such a manner that the pharmacological effects of the two agents are simultaneously exhibited in the patient. Thus, co-administration does not require that a single pharmaceutical composition, the same dosage form, or even the same route of administration be used for administration of both of the compound of the present invention and the other agent or that the two agents be administered at precisely the same time. However, co-administration will be accomplished most conveniently by the same dosage form and the same route of administration, at substantially the same time. Obviously, such administration most advantageously proceeds by delivering both active ingredients simultaneously in a novel pharmaceutical composition in accordance with the present invention.

In some embodiments, in addition to the administration of an ophthalmic pharmaceutical composition, the present method comprises oral administration of an effective amount of the active ingredient (e.g., a rifamycin compound). Suitable oral formulations of the rifamycin compound selected from the group consisting of rifampicin, rifabutin, rifapentine, and rifaximin are known in the present technical field. Thus, the methods include a combination therapy whereby the active ingredient (e.g., rifamycin compound) is administered by to the patient via any suitable mode of administration, including topical, ocular, oral, subcutaneous, mucosal, intradermal, intranasal, buccal, sublingual, pulmonary or the like.

The effective amount of the rifamycin compound is an amount required to produce a protective effect in vitro or in vivo. In some embodiments the effective amount in vitro is from about 0.1 pM to about 1 mM, or about from 0.1 nM to about 1 mM. In some embodiments the effective amount in vitro is from about 1 pM to about 5 pM, about 5 pM to about 10 pM, about 10 pM to about 50 pM, about 50 pM to about 100 pM, about 0.1 nM to about 0.5 nM, about 0.5 nM to about 1.0 nM, about 1.0 nM to about 5.0 nM, about 5.0 nM to about 10 nM, about 10 nM to about 50 nM, about 50 nM to about 100 nM, about 100 nM to about 500 nM, about 500 nM to about 1 μM, about 1 μM to about 10 μM, about 10 μM to about 100 μM, or about 500 nM to about 1 mM. In some embodiments, the effective amount for an effect in vivo is about 0.1 mg to about 100 mg, or preferably, from about 1 mg to about 50 mg, or more preferably, from about 0.07 mg to about 20 mg per kg/day, or about 0.02 mg to about 0.07 mg/kg/day, about 0.07 mg to about 0.2 mg/kg/day, about 0.2 mg to about 0.7 mg/kg/day, about 0.7 mg to about 2 mg/kg/day, about 2 mg to about 7 mg/kg/day, about 7 mg to about 20 mg/kg/day, or about 1 mg to about 12 mg/kg/day. In some other embodiments, the effective amount in vivo is from about 10 mg/kg/day to about 100 mg/kg/day, about 20 mg/kg/day to about 90 mg/kg/day, about 30 mg/kg/day to about 80 mg/kg/day, about 40 mg/kg/day to about 70 mg/kg/day, or about 50 mg/kg/day to about 60 mg/kg/day. In some embodiments, the effective amount in vivo is from about 1 mg/kg/day to about 5 mg/kg/day. In some embodiments, the effective amount in vivo is from about 6 mg/kg/day to about 12 mg/kg/day. In one embodiment, the effective amount in vivo is about 3 mg/kg/day. In another embodiment, the effective amount in vivo is about 6 mg/kg/day. In another embodiment, the effective amount in vivo is about 12 mg/kg/day. In further some other embodiments, the effective amount in vivo is from about 100 mg/kg/day to about 1000 mg/kg/day.

Besides, the dose applied to an animal can be converted to the dose applied to a human (human equivalent dose) with reference to the guidance regarding initial doses of medicaments of U.S. FDA.

### Composition

In one aspect, provided herein is a composition comprising an effective amount of a rifamycin compound, or a pharmaceutically acceptable salt thereof, and a pharmaceutically acceptable carrier, preferably, an ophthalmic pharmaceutical composition (hereinafter referred to as an “ophthalmic composition”).

The rifamycin class of antibiotic was originally isolated from cultures of Streptomyces mediterranei. Due to the large number of available analogues and derivatives generated synthetically, rifamycin has been widely utilized in the elimination of pathogenic bacteria that have become resistant to commonly used antibiotics.

Rifamycin is mainly effective against mycobacteria, and are therefore used to treat chronic infections including tuberculosis (TB), leprosy and Mycobacterium avium complex (MAC) infections. Along with its pulmonary effects, TB is also known to affect other organs including ocular tissues. Therefore, the effects of rifamycin on ocular TB and other ocular disorders have been studied. When chronic administration of rifampicin in HCV patients was monitored, it was observed that alpha fetoprotein which is the liver cancer marker was strongly inhibited. Rifampicin exhibits strong neovascularization effects whereby major neovascularization genes such as VEGF, HGF etc. are strongly inhibited.

Examples of suitable rifamycin compounds may include rifampicin (rifampin), rifabutin, rifapentine, rifalazil and rifaximin, or a pharmaceutically acceptable salt thereof, or a derivative thereof. Syntheses of simple rifamycin derivatives is well known in the present technical field, for example, the synthesis of rifampin (U.S. Pat. No. 3,342,810), rifabutin (U.S. Pat. No. 4,219,478), and rifalazil (U.S. Pat. No. 4,983,602) are known in the present technical field and incorporated herein by reference.

In some embodiments, provided herein is a composition comprising an effective amount of rifamycin compound, preferably, an ophthalmic composition or an eye drop composition, or a pharmaceutically acceptable salt thereof, and an inactive, non-eye-irritating, nontoxic eye drop carrier. Such a carrier is well known, and commonly referred to in, for example, the Physician's Desk Reference for Ophthalmology (1982 Edition, published by Medical Economics Company, Inc., Oridell, New Jersey), wherein a large number of sterile ophthalmologic ocular solutions are reported.

In the present invention, the following substances may be included, but are not limited thereto: benzoic acid (benzoate), ethylenediaminetetraacetic acid, benzalkonium chloride, thimerosal, sodium thiosulfate, citric acid (citrate), glutamic acid (glutamate), chlorobutanol, benzododecinium bromide, sorbic acid, sodium dehydroacetate, magnesium chloride, sodium carbonate, diluted hydrochloric acid, geraniol, acetate, sodium bicarbonate, and potassium iodide.

In some embodiments, examples of a composition including an ophthalmic composition described herein may include eye drops, solutions, ocular solutions such as contact lens solutions, suspensions, gels, creams, and ointments, which are intended for ophthalmic use. In some embodiments, the eye drops are administered with an eye dropper. In some embodiments, the ophthalmic composition is in the form of a topical eye drop. In one embodiment, the ophthalmic composition is a solution. In another embodiment, the ophthalmic composition is a suspension.

In some embodiments, the composition including an ophthalmic composition is included in various drug delivery systems known in the present technical field. For example, the composition can be included in an ocular solution used to clean, preserve, soak or immerse contact lenses. Soft contact lenses comprising a body formed of a hydrophilic gel material, such as 2-hydroxyethyl methacrylate (HEMA), and ocular solutions for cleaning or soaking them are known in the present technical field and are commercially available. In one embodiment, the ophthalmic composition is incorporated in an ocular solution used to soak and swell the hydrophilic gel containing lenses. The lens imbibes the solution comprising the active ingredient and slowly releases it upon insertion in the eye. Thus, therapeutically effective doses of the active ingredient, i.e. rifamycin compounds, are delivered to the patient wearing the contact lens. In one embodiment, contact lenses, after use in the eye can be soaked again in the ocular solution comprising a therapeutic level of the active ingredient to replenish the drug. In some embodiments, the active ingredient can be dispersed in to the matrix of the lens via the polymerization vehicle.

In some embodiments, the concentration of the rifamycin compound applied in the present description can be appropriately determined in the range of about 0.0001% by weight to about 50% by weight. Herein, the “% by weight” can be indicated as “g/100 mL of the composition” or “weight/volume (w/v).”

In one embodiment, the lower limit of the concentration of the rifamycin compound can be selected from the range of about 0.0001% by weight to about 50% by weight, and for example, can be set to be about 0.0001% by weight, 0.0005% by weight, 0.001% by weight, or 0.005% by weight (but is not limited to such % by weight). On the other hand, the upper limit of the concentration of the rifamycin compound can be selected from the above-described range, and for example, can be set to be about 50% by weight, 40% by weight, 30% by weight, or 20% by weight (but is not limited to such % by weight).

In one embodiment of the present invention, provided is a composition including an ophthalmic composition, in which the concentration of the rifamycin compound is about 0.0001% by weight to about 0.0005% by weight, about 0.0005% by weight to about 0.001% by weight, about 0.001% by weight to about 0.005% by weight, about 0.005% by weight to about 0.01% by weight, about 0.01% by weight to about 50% by weight, about 0.01% by weight to about 0.05% by weight, about 0.05% by weight to about 0.1% by weight, about 0.05% by weight to about 40% by weight, about 0.1% by weight to about 0.25% by weight, about 0.1% by weight to about 30% by weight, about 0.25% by weight to about 0.5% by weight, about 0.5% by weight to about 20% by weight, about 0.5% by weight to about 1.0% by weight, about 1.0% by weight to about 2.0% by weight, about 2.0% by weight to about 5.0% by weight, about 1.0% by weight to about 10% by weight, about 1.5% by weight to about 5% by weight, about 2.0% by weight to about 3.0% by weight, about 5.0% by weight to about 10.0% by weight, and a range between any given two values from these values, or a value lower than any given value from these values. In some embodiments, the present ophthalmic composition comprises a rifamycin compound in a concentration of about 50% by weight, 30% by weight, 20% by weight, 10% by weight, about 8% by weight, about 7% by weight, about 5% by weight, about 4% by weight, about 3.5% by weight, about 3% by weight, about 2.5% by weight, about 2% by weight, about 1.5% by weight, about 1% by weight, about 0.5% by weight, about 0.25% by weight, about 0.1% by weight, about 0.05% by weight, about 0.01% by weight, about 0.005% by weight, about 0.002% by weight, about 0.001% by weight, about 0.0005% by weight, or about 0.0001% by weight.

In some embodiments, the composition including an ophthalmic composition comprises a vehicle. Examples of a suitable vehicle for the present ophthalmic compositions may include, but are not limited to, purified water, vegetable oils (e.g., olive oil, castor oil, sesame oil, etc.), and liquid paraffin.

In some embodiments, the composition including an ophthalmic composition further comprises one or more tonicity adjusting agents. In some embodiments, the tonicity adjusting agent is a saline. Suitable tonicity adjusting agents are known in the present technical field, and examples of the tonicity adjusting agent may include, but are not limited to, sodium chloride, potassium chloride, buffer salts, dextrin, glycerin, propylene glycol, and mannitol.

In some embodiments, the composition including an ophthalmic composition optionally comprises one or more surfactants. In some embodiments, a nonionic surfactant aids dispersion of the active ingredient (e.g., a rifamycin compound) in a suspension, and improves the transparency of the solution. Suitable surfactants are known in the present technical field, and examples of the surfactant may include, but are not limited to, sorbitan ether esters of oleic acid (e.g., polysorbate 80 or Tween 20 and 80), polyoxyethylene castor oil, polyoxyethylene hydrogenated castor oil, cremophor, sodium alkylbenzene sulfonate, glycerol, lecithin, sucrose ester, polyoxyethylene-alkyl ether, polyoxyl stearate, polyoxyl 40 stearate, polyethylene glycol monostearate, polymers of oxyethylated octyl phenol (tyloxapol), propylene glycol, benzyl alcohol, macrogol, cyclodextrin, dibutylhydroxytoluene, sorbitol, trometamol, propylene glycol, mannitol, and polyoxyethylene polyoxypropylene glycol (e.g. polyoxyethylene(160) polyoxypropylene(30) glycol, or polyoxyethylene(200) polyoxypropylene(70) glycol), or combinations thereof. In some embodiments, the present ophthalmic composition comprises polysorbate 80, polyoxyethylene hydrogenated castor oil, lecithin, or a combination thereof.

In addition, in the present invention, the ophthalmic composition may comprise an ionic surfactant. Examples of the ionic surfactant may include, but are not limited to, alkyl diaminoethyl glycine hydrochloride solution, benzododecinium bromide, benzalkonium chloride, benzethonium chloride, sodium polystyrene sulfonate, benzoic acid (benzoate), ethylenediaminetetraacetic acid, thimerosal, sodium thiosulfate, citric acid (citrate), glutamic acid (glutamate), benzododecinium bromide, sorbic acid, sodium dehydroacetate, and acetate.

In some embodiments, the ophthalmic composition may not comprise the ionic surfactant at all, or may comprise the ionic surfactant in the range of, for example, about 0.00001% by weight to about 50% by weight. In some embodiments, the ionic surfactant may be comprised in the ophthalmic composition, in a concentration of 0.00005% or less, or in a high concentration of more than 0.00005%.

In some embodiments, with regard to the amount of a surfactant in the present composition, the lower limit can be set to be about 0.00001% by weight, about 0.0001% by weight, or about 0.001% by weight, whereas the upper limit can be set to be about 50% by weight, about 40% by weight, about 30% by weight, about 20% by weight, or about 10% by weight (but are not limited to such % by weight). The appropriate range of the amount of the surfactant is set from these upper and lower limit values. For example, the amount of a nonionic surfactant in the present composition is about 0.00001% by weight to about 50% by weight, about 0.0001% by weight to about 50% by weight, about 0.001% by weight to about 50% by weight, about 0.01% to about 50% by weight, about 0.1% to about 50% by weight, about 0.1% to about 40% by weight, about 0.1% to about 30% by weight, about 1% to about 20% by weight, or about 2% to about 10% by weight.

In some embodiments, the present ophthalmic composition comprises a surfactant in an amount of about 0.00001% by weight to about 50% by weight, about 0.00005% by weight to about 50% by weight, about 0.0001% by weight to about 50% by weight, about 0.001% by weight to about 50% by weight, about 0.01% by weight to 20% by weight, about 0.01% by weight to 15% by weight, about 0.15% by weight to 10% by weight, about 0.2% by weight to 5% by weight, about 0.25% by weight to 3% by weight, about 0.3% by weight to 2% by weight, about 0.1% by weight to 20% by weight, about 1% by weight to 10% by weight, about 2% by weight to 10% by weight, about 2% by weight to 8% by weight, about 2% by weight to 5% by weight, about 5% by weight to 10% by weight, about 5% by weight to 20% by weight, and a range between any given two values from these values, or a value lower than any given value from these values.

In the present invention, the concentration of the surfactant can be adjusted in the range of up to the first, second, third, fourth, or fifth decimal place. For example, the concentration of the surfactant can be set, as appropriate, in the range of 0.00001% to 50.00% by weight, 0.0001% to 50.00% by weight, 0.001% to 50.00% by weight, 0.01% to 50.00% by weight, or 0.10% to 50.00% by weight. Besides, there is an aspect in which the surfactant may not be comprised in the composition of the present invention. The conditions for such an aspect will be described later.

In some embodiments, the present ophthalmic composition comprises a surfactant selected from polysorbate 80, Tween 80, Tween 20, polyoxyethylene hydrogenated castor oil, polyoxyethylene alkyl ether, and lecithin, or a combination thereof.

In some embodiments the composition including an ophthalmic composition may optionally comprise a stabilizer or an antioxidant. Suitable stabilizers and antioxidants are known in the present technical field, and examples thereof may include, but not limited to, ascorbate, ascorbic acid, isoascorbic acid, glutathione sodium bisulfate, sodium metabisulfite, acetyl cysteine, 8-hydroxy quinoline, thiourea, thioglycolate, thiosulfate, tocopherol, EDTA, sodium formaldehyde sulfoxylate dihydrate, and a combination thereof. In some embodiments, the present ophthalmic composition comprises an antioxidant or a stabilizer in an amount of about 0.00001% by weight to about 50% by weight, about 0.00005% by weight to about 50% by weight, about 0.0001% by weight to about 50% by weight, about 0.001% by weight to about 50% by weight, about 0.01% by weight to 20% by weight, about 0.1% by weight to 15% by weight, about 0.15% by weight to 10% by weight, about 0.2% by weight to 5% by weight, about 0.25% by weight to 3% by weight, about 0.3% by weight to 2% by weight, about 0.1% by weight to 20% by weight, about 1% by weight to 10% by weight, about 2% by weight to 10% by weight, about 2% by weight to 8% by weight, about 2% by weight to 5% by weight, about 5% by weight to 10% by weight, about 5% by weight to 20% by weight, and a range between any given two values from these values, or a value lower than any given value from these values. In some embodiments, the present ophthalmic composition comprises an antioxidant or a stabilizer in an amount of about 0.01% by weight to 10% by weight.

In some embodiments, the composition including an ophthalmic composition may optionally comprise a lubricant. Examples of suitable lubricants may include, but are not limited to, glycerol, hydroxypropylmethyl cellulose, carboxy propylmethyl cellulose, sorbitol, polyvinyl pyrrolidone, polyethylene glycol, polyvinyl acetate, and a combination thereof. In some embodiments, the present ophthalmic composition comprises a lubricant in an amount of about 0.00001% by weight to about 50% by weight, about 0.00005% by weight to about 50% by weight, about 0.0001% by weight to about 50% by weight, about 0.001% by weight to about 50% by weight, about 0.01% by weight to 20% by weight, about 0.1% by weight to 15% by weight, about 0.15% by weight to 10% by weight, about 0.2% by weight to 5% by weight, about 0.25% by weight to 3% by weight, about 0.3% by weight to 2% by weight, about 0.1% by weight to 20% by weight, about 1% by weight to 10% by weight, about 2% by weight to 10% by weight, about 2% by weight to 8% by weight, about 2% by weight to 5% by weight, about 5% by weight to 10% by weight, about 5% by weight to 20% by weight, and a range between any given two values from these values, or a value lower than any given value from these values. In some embodiments, the present ophthalmic composition comprises about 0.01% by weight to 10% by weight of the lubricant.

In some embodiments, the composition including an ophthalmic composition may optionally comprise a deturgescent agent. Examples of suitable deturgescent agents may include, but are not limited to, low or high molecular weight polysaccharides, such as, for example, dextran, dextran sulfate, polyvinyl pyrrolidone, polyethylene glycol, polyvinyl acetate, hydroxypropylmethyl cellulose, carboxymethyl cellulose, carboxypropylmethyl cellulose, dextrose, sucrose, other sugars, and a combination thereof. Any given dextran having a suitable molecular weight, including dextran 40, dextran 70, and/or dextran 500, or a mixture thereof may be used. In some embodiments, the present ophthalmic composition comprises a deturgescent agent in an amount of about 0.00001% by weight to about 50% by weight, about 0.00005% by weight to about 50% by weight, about 0.0001% by weight to about 50% by weight, about 0.001% by weight to about 50% by weight, about 0.01% by weight to 20% by weight, about 0.1% by weight to 15% by weight, about 0.15% by weight to 10% by weight, about 0.2% by weight to 5% by weight, about 0.25% by weight to 3% by weight, about 0.3% by weight to 2% by weight, about 0.1% by weight to 20% by weight, about 1% by weight to 10% by weight, about 2% by weight to 10% by weight, about 2% by weight to 8% by weight, about 2% by weight to 5% by weight, about 5% by weight to 10% by weight, about 5% by weight to 20% by weight, and a range between any given two values from these values, or a value lower than any given value from these values. In some embodiments, the present ophthalmic composition comprises about 0.01% by weight to 10% by weight of the deturgescent agent.

In some embodiments, the composition including an ophthalmic composition may further comprise one or more viscosity imparting agents. In some embodiments, the viscosity imparting agent increases the viscosity of an ophthalmic solution and a suspension. In some embodiments, the viscosity imparting agent increases an ocular contact time, thereby decreasing the drainage rate. In some embodiments, the viscosity imparting agent increases mucosa adhesion and ocular bioavailability, and/or imparts lubricating effects. Examples of suitable viscosity imparting agents may include, but are not limited to, carboxyvinyl polymers (e.g., Carbopol 934P or 974P), cellulosic polymers (e.g., carboxymethyl cellulose, hydroxypropylmethyl cellulose, hydroxyethyl cellulose or the like), polysaccharides (e.g., xanthan gum), polyvinyl pyrrolidone, polyvinyl alcohol, chondroitin sulfate, lanolin, hyaluronic acid, propylene glycol, and a combination thereof. In some embodiments, the present ophthalmic composition comprises a viscosity imparting agent in an amount of about 0.00001% by weight to about 50% by weight, about 0.00005% by weight to about 50% by weight, about 0.0001% by weight to about 50% by weight, about 0.001% by weight to about 50% by weight, about 0.01% by weight to 20% by weight, about 0.1% by weight to 15% by weight, about 0.15% by weight to 10% by weight, about 0.2% by weight to 5% by weight, about 0.25% by weight to 3% by weight, about 0.3% by weight to 2% by weight, about 0.5% by weight to 2% by weight, about 0.1% by weight to 20% by weight, about 1% by weight to 10% by weight, about 2% by weight to 10% by weight, about 2% by weight to 8% by weight, about 2% by weight to 5% by weight, about 5% by weight to 10% by weight, about 5% by weight to 20% by weight, and a range between any given two values from these values, or a value lower than any given value from these values. In some embodiments, the present ophthalmic composition comprises about 0.01% by weight to 10% by weight of the viscosity imparting agent.

In some embodiments, the composition including an ophthalmic composition may comprise one or more phospholipid compounds. Suitable phospholipids are known in the present technical field, and examples thereof may include, but are not limited to, small alkyl chain phospholipids, phosphatidylcholine, egg phosphatidylcholine, soybean phosphatidylcholine, dipalmitoylphosphatidylcholine, soy phosphatidylglycerol, egg phosphatidylglycerol, distearoylphosphatidylglycerol, dimyristoylphosphatidylcholine, distearoylphosphatidylcholine, dilaurylphosphatidylcholine, 1-myristoyl-2-palmitoylphosphatidylcholine, 1-paimitoyl-2-myristoylphosphatidylcholine, 1-palmitoyl-2-stearoylphosphatidylcholine, 1-stearoyl-2-palmitoylphosphatidylcholine, dioleoylphosphatidylcholine, 1-palmitoyl-2-oleoylphosphatidylcholine, 1-oleoyl-2-palmitylphosphatidylcholine, dioleoylphosphatidylethanolamine, dilauroylphosphatidylglycerol, phosphatidylserine, phosphatidylethanolamine, phosphatidylinositol, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, phosphatidic acid, dimyristoylphosphatidic acid, dipaimitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, sphingomyelin, sphingolipids, brain sphingomyelin, dipalmitoylsphingomyelin, distearoylsphingomyelin, galactocerebroside, gangliosides, cerebrosides, phosphatidylglycerol, phosphatidic acid, lysolecithin, lysophosphatidylethanolamine, cephalin, cardiolipin, dicetylphosphate, distearoyl-phosphatidylethanolamine, or combinations thereof. The phospholipid may also be a derivative or analogue of any of the above-described phospholipids. In some embodiments, the present ophthalmic composition comprises a phospholipid compound(s) in an amount of about 0.00001% by weight to about 50% by weight, about 0.00005% by weight to about 50% by weight, about 0.0001% by weight to about 50% by weight, about 0.001% by weight to about 50% by weight, about 0.01% by weight to 20% by weight, about 0.1% by weight to 15% by weight, about 0.15% by weight to 10% by weight, about 0.2% by weight to 5% by weight, about 0.25% by weight to 3% by weight, about 0.3% by weight to 2% by weight, about 0.1% by weight to 20% by weight, about 1% by weight to 10% by weight, about 2% by weight to 10% by weight, about 2% by weight to 8% by weight, about 2% by weight to 5% by weight, about 5% by weight to 10% by weight, or about 5% by weight to 20% by weight. In some embodiments, the present ophthalmic composition comprises about 0.01% by weight to 10% by weight of the phospholipid compound(s).

In some embodiments, the composition including an ophthalmic composition may optionally comprise a preservative agent. Examples of the preservative agent may include, but are not limited to, imidazolidinyl urea, methylparaben, propylparaben, phenoxyethanol, disodium EDTA, benzalkonium chloride, thimerosal, chlorobutanol sorbic acid, and a combination thereof. In some embodiments, the present ophthalmic composition comprises a preservative agent in an amount of about 0.00001% by weight to about 50% by weight, about 0.00005% by weight to about 50% by weight, about 0.0001% by weight to about 50% by weight, about 0.001% by weight to about 50% by weight, about 0.01% by weight to 20% by weight, about 0.1% by weight to 15% by weight, about 0.15% by weight to 10% by weight, about 0.2% by weight to 5% by weight, about 0.25% by weight to 3% by weight, about 0.3% by weight to 2% by weight, about 0.1% by weight to 20% by weight, about 1% by weight to 10% by weight, about 2% by weight to 10% by weight, about 2% by weight to 8% by weight, about 2% by weight to 5% by weight, about 5% by weight to 10% by weight, or about 5% by weight to 20% by weight. In some embodiments, the present ophthalmic composition comprises about 0.01% by weight to 10% by weight of the preservative agent.

Herein, the above-described preservative agent can be used also as an ionic surfactant.

In some embodiments, the composition including an ophthalmic composition may optionally comprise one or more buffer agents for maintaining the pH of the composition in a range generally acceptable for eye drop compositions. In some embodiments, the pH of the present composition is, for example, a pH value of about 1 to 12, 2 to 12, 3 to 9, or 3 to 7.5. Depending on the combination of a surfactant and a buffer solution, the pH of the composition is adjusted, in detail, up to the first, second, or third decimal place.

For example, when the pH is adjusted to the third decimal place, the lower limit value is, for example, 1.000, 2.000, 3.000, 4.000, 5.000, etc., whereas the upper limit value is, for example, 12.000, 11.000, 10.000, 9.000, 8.000, etc.

Moreover, examples of the used buffer solution may include, but are not limited to: acids, such as acetic acid, boric acid, citric acid, lactic acid, phosphoric acid, hydrochloric acid-potassium chloride, glycine, aconitic acid, citric acid-phosphoric acid, succinic acid, phthalic acid, maleic acid, cacodylic acid, tris(trishydroxymethylaminomethane), barbituric acid, borax, 2-amino-2-methyl-1, 3-propanediol (Ammediol), sodium carbonate-sodium bicarbonate, HEPES (4-(2-hydroxyethyl)-1-piperazineethanesulfonic acid), ACES (N-(2-acetamido)-2-aminoethanesulfonic acid), ADA (N-(2-acetamido)iminodiacetic acid), BES (N,N-bis(2-hydroxyethyl)-2-aminoethanesulfonic acid), Bicine (N,N-bis(2-hydroxyethyl)glycine), Bis-Tris (bis(2-hydroxyethyl)iminotris(hydroxymethyl)methane), CAPS (N-cyclohexyl-3-aminopropanesulfonic acid), CAPSO (N-cyclohexyl-2-hydroxy-3-aminopropanesulfonic acid), CHES (N-cyclohexyl-2-aminoethanesulfonic acid), DIPSO (3-[N,N-bis(2-hydroxyethyl)amino]-2-hydroxypropanesulfonic acid), EPPS (3-[4-(2-hydroxyethyl)-1-piperazinyl]propanesulfonic acid), HEPES-Na (sodium 2-[4-(2-hydroxyethyl)-1-piperazinyl]ethanesulfonate), HEPPSO (2-hydroxy-3-[4-(2-hydroxyethyl)-1-piperazinyl]propanesulfonic acid, monohydrate), MES (2-morpholinoethanesulfonic acid, monohydrate), MOPS (3-morpholinopropanesulfonic acid), MOPSO (2-hydroxy-3-morpholinopropanesulfonic acid), PIPES (piperazine-1, 4-bis(2-ethanesulfonic acid)), POPSO (piperazine-1, 4-bis(2-hydroxy-3-propanesulfonic acid), dihydrate), TAPSO (2-hydroxy-N-tris(hydroxymethyl)methyl-3-aminopropanesulfonic acid), TES (N-tris(hydroxymethyl)methyl-2-aminoethanesulfonic acid), Tricine (N-[tris(hydroxymethyl)methyl]glycine), and hydrochloric acid; bases, such as sodium hydroxide, sodium phosphate, sodium borate, sodium citrate, sodium acetate, and sodium lactate; and buffer agents, such as citrate/dextrose, sodium bicarbonate, and ammonium chloride, and citrate, phosphate, borate, bicarbonate, sodium salt and potassium, comprising a combination thereof.

Examples of the Tris buffer solution used in the present invention may include, but are not limited to, a Tris-HCl buffer solution, a TE buffer solution (for example, having a composition of 10 mM Tris/Tris-HCl and 1 mM EDTA), a TAE buffer solution (for example, having a composition of 40 mM Tris/Tris-acetate and 1 mM EDTA), a TBE buffer solution (for example, having a composition of 89 mM Tris/Tris-borate and 2 mM EDTA), and TBS (for example, having a composition of 10 mM Tris/Tris-HCl and 150 mM NaCl).

The above-described acid, base, and buffer agent are comprised in the composition, in an amount necessary for maintaining a pH value, at which the rifamycin compound contained in the composition is dissolved in an aqueous vehicle, or the pH of the composition within a pH range acceptable for eyes.

The composition including an ophthalmic composition may additionally comprise a suitable diluent known in the present technical field. In some embodiments, the diluent is an isotonic eye treatment carrier, which is buffered to a suitable pH value, for example, in the pH range of from about 4.0 to about 8.0, and contains effective amounts of a wetting agent and an antibacterial agent.

When structural ingredients of the above-described composition including an ophthalmic composition are present, the structural ingredients may be incorporated into the composition in a concentration range of about 0.00001% by weight, about 0.00005% by weight, about 0.0001% by weight, about 0.001% by weight, about 0.01% by weight, about 0.02% by weight, about 0.05% by weight, about 0.1% by weight, about 0.5% by weight, about 1.0% by weight, about 2% by weight, about 5% by weight, about 10.0% by weight, about 15.0% by weight, about 20.0% by weight, about 30.0% by weight, about 40.0% by weight, about 50.0% by weight, about 60.0% by weight, about 70.0% by weight, or about 90.0% by weight, and in a range between any given two values from these values, or in a concentration lower than any one of these values.

The composition including an ophthalmic composition can be produced by methods known in the present technical field. For example, the active ingredient, namely, the rifamycin compound is dissolved in purified water or a saline, and a surfactant is then added thereto and mixed therein. Further additives, such as, for example, an isotonic agent such as sodium chloride or glycerin, a buffer agent such as sodium phosphate or sodium borate, a pH adjuster such as diluted hydrochloric acid or sodium hydroxide, a preservative agent such as potassium sorbate, and an antioxidant such as tocopherol or ascorbic acid, are optionally added to the mixture to obtain an ophthalmic composition.

The composition including an ophthalmic composition is tested for various physicochemical, in vitro, and in vivo properties. Transparency is measured using a visual method and a fluorescent microscopic method. Moreover, in order to ensure that the ophthalmic solution does not contain foreign particles, the presence of a fine particulate matter is measured. A light obscuration method or a microscopic method is used for counting and/or for measuring the particle size. The isotonicity and pH of the composition are tested.

The content of a drug in the present ophthalmic composition is evaluated by suitable analytical methods such as UV and HPLC. In addition, the composition is also tested for preservative agent effectiveness, stability, and effective retention period, according to standard guidelines. The present composition can be further subjected to sterilization using various sterilization methods known in the present technical field.

In the composition of the present invention, the rifamycin compound or a pharmaceutically acceptable salt thereof is stably dissolved in the above-described aqueous solution formulation.

The term “stable” means that the rifamycin compound or a pharmaceutically acceptable salt thereof is dissolved in the aqueous solution formulation, and then, precipitation or the like does not occur after a certain period of time has passed. Examples of the certain period of time may include 2 hours, 5 hours, 10 hours, whole day and night, 1 day, 2 days, 3 days, 1 week, 2 weeks, 3 weeks, 1 month, 2 months, 3 months, and 6 months. In addition, the temperature for maintaining stability is, for example, 0° C. to 40° C. (the upper limit: 40° C.; the lower limit: 0° C.), and the temperature can be set, as appropriate, in the above temperature range. The temperature may be a constant temperature, or may also be in a constant temperature range. Specific examples of the temperature (range) for maintaining stability may include 0° C., 4° C., 10° C., 15° C., 20° C., 25° C., 30° C., 35° C., 37° C., 40° C., 4° C. to 40° C., 4° C. to 37° C., 4° C. to 30° C., 4° C. to 25° C., 4° C. to 20° C., 4° C. to 10° C., 10° C. to 37° C., 10° C. to 30° C., 10° C. to 25° C., 10° C. to 20° C., 20° C. to 25° C., 20° C. to 37° C., 25° C. to 37° C., and 25° C. to 30° C.

As described above, with regard to the composition of the present invention, by specifically studying the pH of the aqueous solution formulation and the concentration of the nonionic surfactant, a range in which the rifamycin compound is stably dissolved for a long period of time was discovered.

In the present invention, whether or not the rifamycin compound is dissolved in an aqueous vehicle is used as an indicator, and the pH and the nonionic surfactant concentration in which the compound is dissolved, and the pH and the nonionic surfactant concentration in which the compound is not dissolved, are plotted in an XY coordinate (two-dimensional coordinate). The plots serving as boundaries for dissolution and non-dissolution are successively connected with one another using a line, so that a region in which the rifamycin compound is dissolved in an aqueous vehicle (referred to as a “soluble region”) and a region in which the rifamycin compound is not dissolved in an aqueous vehicle (referred to as an “insoluble region”) can be displayed. The phrase “to successively connect” means that a certain plot is connected with a plot adjacent to the certain plot (wherein the plots mean two plots in a soluble region, or two plots in an insoluble region).

For example, in the case of taking the above-described XY coordinate as an example, the X-axis is set to be pH and the Y-axis is set to be the nonionic surfactant concentration, and the pH and the nonionic surfactant concentration obtained when the rifamycin compound is dissolved in an aqueous vehicle are plotted as coordinates (referred to as a “soluble plot”). In addition, the pH and the nonionic surfactant concentration obtained when the rifamycin compound is not dissolved in an aqueous vehicle are plotted as coordinates (referred to as an “insoluble plot”). By plotting a plurality of coordinates under several conditions, a region in which soluble plots are gathered, and a region in which insoluble plots are gathered, can be displayed. The plot that forms the boundary portion of the two regions can be represented by (X,Y)=(m,n) (wherein m represents pH, and n represents the nonionic surfactant concentration), and specific numerical values will be described in the Examples later. It is to be noted that (X,Y)=(m,n) may indicate either soluble plots or insoluble plots. By connecting adjacent soluble plots or adjacent insoluble plots with each other, a boundary between the soluble region and the insoluble region can be expressed as a line.

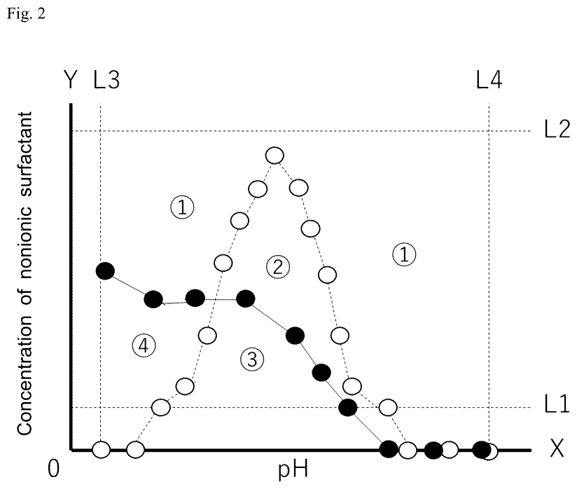

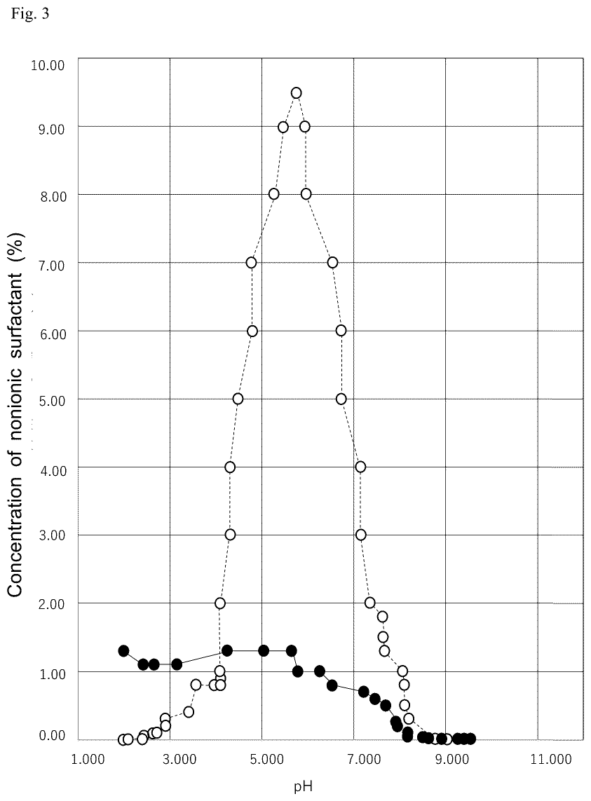

FIG. 2 is a view showing the relationship between the pH and the nonionic surfactant concentration, in which the rifamycin compound is dissolved in an aqueous vehicle.

In FIG. 2, the X-axis indicates pH, and the Y-axis indicates the nonionic surfactant concentration. The broken lines L1 and L2 indicate the lower limit value and the upper limit value of the nonionic surfactant concentration, respectively, whereas the broken lines L3 and L4 indicate the lower limit value and the upper limit value of the pH, respectively.

In the present invention, as a boundary between the soluble region and the insoluble region, multiple patterns can be obtained depending on the buffer solution used.

Among soluble plots, when plots constituting the boundary with insoluble plots are connected with one another using a line, the line formed by connecting soluble plots with one another, when a buffer solution other than a boric acid buffer solution is used as a buffer solution, is shown with open circles (∘) in the graph of FIG. 3, and the line has a rocket shape (a line or curve). As with the above, the line formed by connecting soluble plots constituting the boundary with insoluble plots, when a boric acid buffer solution is used as a buffer solution, is shown in filled circles (•) in the graph of FIG. 3.

In FIG. 2, when the regions divided in each graph are represented by Region 1 (number 1 enclosed with a circle) to Region 4 (number 4 enclosed with a circle), Region 1 represents both a soluble plot when a boric acid buffer solution is used and a soluble plot when a buffer solution other than the boric acid buffer solution is used, and Region 1 is a soluble region that is commonly dissolved in a buffer solution, regardless of the type of the used buffer solution.

Region 2 (number 2 enclosed with a circle) represents a soluble plot when a boric acid buffer solution is used, but it represents an insoluble plot when a buffer solution other than the boric acid buffer solution is used. Thus, Region 2 is a soluble region when a boric acid buffer solution is used, and is an insoluble region when a buffer solution other than the boric acid buffer solution is used.

Region 3 (number 3 enclosed with a circle) represents both an insoluble plot when a boric acid buffer solution is used and an insoluble plot when a buffer solution other than the boric acid buffer solution is used, and Region 3 is an insoluble region that is not commonly dissolved in a buffer solution, regardless of the type of the used buffer solution. Thus, Region 3 becomes an insoluble region, regardless of the type of the used buffer solution.

Region 4 (number 4 enclosed with a circle) represents a soluble plot when a buffer solution other than a boric acid buffer solution is used, but it represents an insoluble plot when the boric acid buffer solution is used. Thus, Region 4 becomes an insoluble region when a boric acid buffer solution is used, and becomes a soluble region when a buffer solution other than the boric acid buffer solution is used.

Besides, in FIG. 2, for the convenience of explanation, two types of graphs are included in a single XY coordinate, and Regions 1 to 4 are displayed. If a single graph is used, independently, each graph shows two regions.

The graph shown with open circles in FIG. 2 depicts a rocket shape (a straight line or curve). The region on this line and the region located outside thereof are composed of Region 1 and Region 4, and they represent a soluble region. Region 2 and Region 3 each represent an insoluble region of a rifamycin compound (including a region in which even if the rifamycin compound is dissolved, then precipitation occurs).

In the graph of a soluble region (•) formed by connecting the plots of the boundary between soluble plots and insoluble plots with one another using a line, when a boric acid buffer solution is used as a buffer solution, the region on this line and the region located outside thereof, namely, a convex outside region (Region 1 and Region 2) represents a soluble region of the rifamycin compound, whereas an inside region enclosed with the graph, namely, a convex inside region (Region 3 and Region 4) represents an insoluble region of the rifamycin compound (including a region in which even if the rifamycin compound is dissolved, then precipitation occurs). The graph (•) almost forms a plateau with a constant surfactant concentration from approximately the lower limit value of the pH to a certain pH value, and thereafter, the graph moves, while falling to the right.

However, in the present invention, the graph obtained with the above-described XY plot values is generalized with a numerical formula, and the site between plots can be represented with an approximate curve, instead of a straight line. Such generalization can be easily carried out by those skilled in the art using numerical formula replacement software or application.

Furthermore, in the present invention, with regard to the above-described XY plot, by using the product (area under the curve: AUC) of the above-described concentration (% by weight) of the nonionic surfactant with the above-described pH, the relationship between the concentration (% by weight) of the rifamycin compound with AUC can be represented by the following approximate equation (1) of higher-order function. Specifically, when the values obtained by setting the x-axis to be the concentration (% by weight) of the rifamycin compound and the y-axis to be AUC are plotted, it can be represented by the following equation:

y_{AUC}=ax²+bx+c (1).

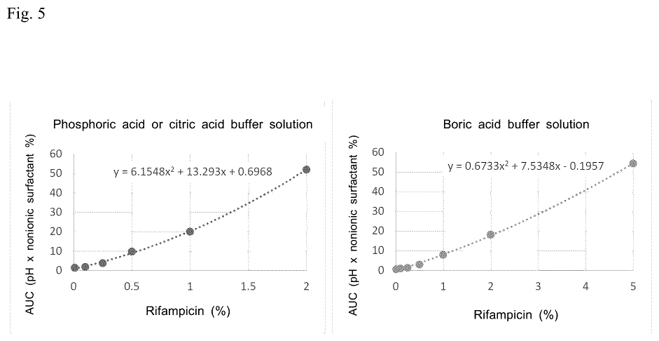

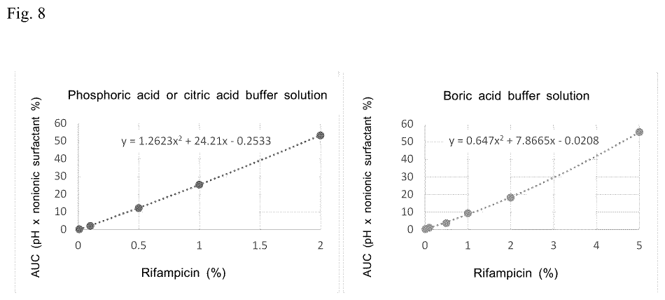

In the above equation (1), y_{AUC} represents the area under the curve, x represents the concentration (% by weight) of the rifamycin compound, and a, b, and c each have a predetermined coefficient or value. For example, a, b, and c have the following relationship: (a, b, c)=(6.1548, 13.293, 0.6968), (0.6733, 7.5348, −0.1957), (1.2623, 24.21, −0.2533) or (0.647, 7.8665, −0.0208) (see FIG. 5 and FIG. 8).

When this equation is satisfied, it can be defined that the rifamycin compound or a pharmaceutically acceptable salt thereof is stably dissolved in an aqueous solution formulation.

Herein, as a method of deriving a straight line or a curve from the plots on the two-dimensional coordinate, for example, a least-squares method or the like may be adopted, and such a method is publicly known to those skilled in the art.

In addition, as described in the Examples later, the relationship between the concentration (% by weight) of the rifamycin compound and the concentration (% by weight) of the nonionic surfactant in each buffer solution and each pH value may also be an approximate relationship, as with the above equation (1). When such an approximate relationship could be obtained, it can be defined that the rifamycin compound or a pharmaceutically acceptable salt thereof is stably dissolved in an aqueous solution formulation. Even in this case, the method of deriving a straight line or a curve from the plots on the two-dimensional coordinate is the same as that described above. For example, in one embodiment of the present invention, the relationship between the concentration of the nonionic surfactant and the concentration of the rifamycin compound can be represented by the following equation (2):

y_{Nis}=ax³+bx²+cx+d (2)

[wherein y_{Nis} represents the concentration of the nonionic surfactant, and x represents the concentration of the rifamycin compound].

In the above equation, a, b, c, and d each represent a predetermined value, and the details thereof will be described in the Examples later. In this case, the above equation (2) can be defined, while dividing into a case where the composition comprises more than 0.00005% of an ionic surfactant, and a case where the composition does not comprise such an ionic surfactant or comprises the ionic surfactant in a concentration of lower than 0.00005%.

The composition of the present invention is tested for various physicochemical, in vitro, and in vivo properties. For example, the composition is evaluated for inhibition of gene expression of neovascularization genes in vitro and protection of retinal ganglion cells, using suitable methods known in the present technical field.

The ophthalmic composition of the present invention has high efficiency and is expected to exhibit high affinity to mucosal tissues, including eye ball. Some embodiments described herein relate to a composition, which delivers a therapeutically effective amount of drug to the systemic circulation via the mucosa. In some embodiments, the composition of the present invention provides advantages over other forms of administration routes (e.g. oral or intravitreal administration, etc.), which include, but are not limited to, avoiding first pass metabolism of drug(s), avoiding irritation of the GI mucosa, avoiding fluctuation in drug levels, predictable and extended duration of activity, minimizing undesirable side effects, suitability for drugs with short half-life and narrow therapeutic index, maintaining steady plasma concentrations of potent drugs, greater patient compliance due to elimination of multiple doses and dosage forms (oral and systemic), no requirement for local anesthesia, no pain associated with injections, the ease of administration, suitability for self-administration, and the ease of terminating of therapy at any point in time.

In some embodiments, provided herein is an ophthalmic composition for use in the production of a medicament for treatment of an ocular or visual disorder. In some embodiments, the ocular disease, disorder or condition is selected from age related macular degeneration (AMD), ocular neovascularization, retinal ganglion cell injury, cicatrization, glaucoma surgery, and brain damage.

In some embodiments, provided herein is a formulation for treatment of an ocular disease, disorder or condition, wherein the formulation comprises one selected from the group consisting of rifampicin, rifabutin, rifapentine, and rifaximin, and at least one pharmaceutically acceptable carrier substance. In some embodiments, the carrier substance comprises an ocular/ophthalmic carrier. Suitable ocular/ophthalmic carrier substances are known in the present technical field, and examples thereof may include solution, gel, and ointment. In some embodiments, the ocular/ophthalmic carrier is a sterilized aqueous solution. In some embodiments, the present formulation further comprises one or more of a surfactant, an antibacterial agent, a pH buffer agent, an antioxidant, a preservative agent, or a combination thereof.

In some embodiments, provided herein is a composition including an ophthalmic composition for treating age-related macular degeneration, and the composition comprises an effective amount of a rifamycin compound selected from the group consisting of rifampicin, rifabutin, rifapentine, and rifaximin.

In some embodiments, provided herein is a composition including an ophthalmic composition for treating retinal neovascularization, and the composition comprises an effective amount of a rifamycin compound selected from the group consisting of rifampicin, rifabutin, rifapentine, and rifaximin.

In some embodiments, provided herein is a composition including an ophthalmic composition for protecting optic nerve cells, and the composition comprises an effective amount of a rifamycin compound selected from the group consisting of rifampicin, rifabutin, rifapentine, and rifaximin.

In some embodiments, provided herein is a composition including an ophthalmic composition for inhibiting brain damage, and the composition comprises an effective amount of a rifamycin compound selected from the group consisting of rifampicin, rifabutin, rifapentine, and rifaximin.

In another embodiment, provided herein is a composition including a stabilized ophthalmic composition comprising a rifamycin compound, wherein the composition is stable for more than three months, preferably more than six months, and more preferably more than twelve months.

### EXAMPLES

Hereinafter, the present invention will be further specifically described in the following examples. However, these examples are not intended to limit the scope of the present invention.

### Example 1

### Eye Drop Formulation Comprising Rifamycin Compound

The active ingredient, namely, rifampicin, rifabutin, rifapentine, rifalazil or rifaximin is dissolved in a saline or water, and a surfactant such as polysorbate 80, Tween 80 or Tween 20 is added to and mixed with the solution. Further, various additives such as glycerin, xanthan gum, hydroxypropylmethyl cellulose (HPMC), a cyclodextrin derivative such as hydroxypropyl-β-cyclodextrin, an isotonic agent such as sodium chloride, potassium chloride or sodium bisulfate, a preservative agent such as disodium EDTA or methylparaben, and an antioxidant such as ascorbic acid are optionally added to and mixed with the solution to form a transparent solution. The thus obtained solution is optionally filtered to remove fine particulate matters, and the pH thereof is then adjusted by adding an acid such as hydrochloric acid or a base such as sodium chloride, thereby obtaining a desired pH value.

### Example 2

As disclosed in Tables 1-3, 16 types of various formulations were prepared for application of topical eye drops comprising rifampicin. The formulations of Example 1A and Example 6A were used in the following studies.

The following eye drop formulations were prepared at room temperature (Tables 1 and 2), and rifampicin was then added to the eye drop formulations to obtain the final concentrations as listed below.

TABLE 1

| | Ex. 1A | Ex. 2A | Ex. 3A | Ex. 4A | Ex. 5A |
|---|---|---|---|---|---|
| pH of added buffer solution | | | | pH 8.7 | pH 8.7 |
| (50 mM boric acid-borax) | | | | | |
| pH of added buffer solution | pH 8.3 | pH 8.3 | pH 8.3 | | |
| (100 mM boric acid-NaOH) | | | | | |
| Final concentration of added | 10 mM | 10 mM | 20 mM | 25 mM | 25 mM |
| buffer solution | | | | | |
| Final pH detected after | pH 7.64 | | pH 7.37 | pH 8.01 | pH 8.30 |
| addition of rifampicin | | | | | |
| NaCl* | 150 mM | 150 mM | 0.9% | 0.9% | 0.9% |
| Tween 80* | 0.5% | 0.5% | 0.5% | 0.5% | 0.5% |
| EDTA* | 0.1% | 0.1% | 0.1% | 0.1% | 0.1% |
| Benzalkonium chloride* | 0.01% | 0.01% | 0.01% | 0.01% | 0.01% |
| Rifampicin* | 0.25% | 0.5% | 0.5% | 1% | 0.5% |

*Final concentration

TABLE 2

| | Ex. 6A | Ex. 7A | Ex. 8A | Ex. 9A | Ex. 10A |
|---|---|---|---|---|---|
| pH of added buffer solution | pH 8.7 | pH 8.7 | pH 8.5 | pH 8.5 | pH 8.3 |
| (50 mM boric acid-borax) | | | | | |
| Final concentration of added | 18 mM | 14 mM | 25 mM | 18 mM | 25 mM |
| buffer solution | | | | | |
| Final pH detected after | pH 8.01 | pH 8.02 | pH 7.81 | pH 7.69 | pH 7.47 |
| addition of rifampicin | | | | | |
| NaCl* | 0.9% | 0.9% | 0.9% | 0.9% | 0.9% |
| Tween 80* | 0.5% | 0.5% | 0.5% | 0.5% | 0.5% |
| EDTA* | 0.1% | 0.1% | 0.1% | 0.1% | 0.1% |
| Benzalkonium chloride* | 0.01% | 0.01% | 0.01% | 0.01% | 0.01% |
| Rifampicin* | 0.5% | 0.5% | 0.5% | 0.5% | 0.5% |

*Final concentration*

Rifampicin was completely dissolved at room temperature in the eye drop formulations of Example 1A and Examples 5A to 8A listed in Tables 1 and 2. Rifampicin was not precipitated in these formulations for several weeks. Rifampicin solutions were preserved at room temperature or in a refrigerator.

The buffer solutions, which were added in Examples 1A to 10A, were 50 mM boric acid-borax or 100 mM boric acid-NaOH. The pH values of these added buffer solutions were listed in Tables 1 and 2. Liquid formulations were prepared by mixing the structural ingredients listed in the tables with one another, and powders of rifampicin were then added into these formulations, followed by mixing for 2 to 3 hours. Rifampicin was mixed into the formulations, and the pH values were then measured. The obtained pH values were listed in the tables.

TABLE 3

| | Ex. 11A | | Ex. 12A | | Ex. 13A | | Ex. 14A | | Ex. 15A | | Ex. 16A | |
|---|---|---|---|---|---|---|---|---|---|---|---|---|
| pH of added buffer solution | | | | | | | | | pH 8.7 | | pH 8.7 | |
| (50 mM boric acid-borax) | | | | | | | | | | | | |
| pH of added buffer solution | pH 8.3 | | pH 8.3 | | pH 8.3 | | pH 8.3 | | | | | |
| (100 mM boric acid-NaOH) | | | | | | | | | | | | |
| Final concentration of added | 10 | mM | 10 | mM | 10 | mM | 10 | mM | 18 | mM | 18 | mM |
| buffer solution | | | | | | | | | | | | |
| Final pH detected after | pH 8.03 | | pH 8.10 | | pH 4.07 | | pH 3.32 | | pH 5.23 | | pH 3.67 | |
| addition of all structural | | | | | | | | | | | | |
| ingredients including | | | | | | | | | | | | |
| rifampicin | | | | | | | | | | | | |
| NaCl* | 150 | mM | 150 | mM | 150 | mM | 150 | mM | 0.90% | | 0.90% | |
| Tween 80* | 0.5% | | 0.5% | | 0.5% | | 0.5% | | 0.5% | | 0.5% | |
| EDTA* | 0.1% | | 0.1% | | 0.1% | | 0.1% | | 0.1% | | 0.1% | |
| Benzalkonium chloride* | 0.01% | | 0.01% | | 0.01% | | 0.01% | | 0.01% | | 0.01% | |
| Rifampicin* | 2.5 | mg/ml | 2.5 | mg/ml | 2.5 | mg/ml | 2.5 | mg/ml | 5 | mg/ml | 5 | mg/ml |

| | | | | |
|---|---|---|---|---|
| Sodium formaldehyde | 0.04 | mg/ml | 0.21 | mg/ml |

| | | | | |
|---|---|---|---|---|
| sulfoxylate dihydrate* | | | | |
| L-ascorbic acid* | 0.25% | 1% | 0.25% | 1% |

*Final concentration

The buffer solution, NaCl, Tween 80, and EDTA as listed in Table 3 were mixed with one another at room temperature in a beaker, and thereafter, rifampicin was added to the mixture and was completely dissolved in these liquid formulations at room temperature. A stock solution of sodium formaldehyde sulfoxylate dihydrate or L-ascorbic acid was added into each formulation, and the pH value of the formulation was then measured (Examples 11A to 16A). The formulations of Example 11A and 12A were stable at room temperature over more than several days, and no precipitates were generated therein. The formulations of Examples 13A to 16A were stable in a refrigerator over more than several days, and no precipitates were generated therein.

### Example 3

Rifampicin was delivered to the retina by application of topical eye drops. A 0.25% Rifampicin eye drop formulation was used in these studies. The eye drop formulation showed good delivery efficiency, and the retinal tissues obtained a micro gram concentration of rifampicin per g of the tissues by the eye drop application.

Four male Sprague-Dawley rats (250 to 300 g) were used to measure the retina exposure level of rifampicin after the application of eye drops. Two rats received 3 drops of the eye drop formulation (0.25% rifampicin), which is shown as Example 1A in Table 1, in each eye under isoflurane sedation. The remaining two rats received the same application, but 10 drops of the eye drops, in each eye under isoflurane sedation. A single drop contained 5 μL of the above-described formulation, and application of each drug drop was carried out with intervals of 30 minutes. After the eye drop application, the retina was excised from each rat under a dissection microscope. In addition, “non-treated” negative controls were used, and the retina was excised from two rats without performing any treatments on the rats. The retina was placed in a 1.5-ml microcentrifuge tube (1 retina/tube), and was fully washed with DPBS. After completion of the washing procedures, the retinal tissues in the microcentrifuge tube were frozen in dry ice, and were then preserved for quantification by LC/MS analysis. Table 4 shows quantification of rifampicin which was extracted from the retinal tissues. Rifampicin was delivered to the retina by the eye drop formulation (0.25% rifampicin) in a dose dependent manner.

TABLE 4
Amount of rifampicin delivered to retina by application
of topical eye drops (0.25% rifampicin)

| | Non-treated | 3 drops | 10 drops |
|---|---|---|---|
| Rifampicin extracted from retina | BDL | 1,288* | 2,939* |
| (Average of n = 3, | | | |
| ng/1 g of tissues) | | | |
| Standard deviation | N/A | 394 | 50.54 |

BDL: Below detection limit
*Evaluated by p value of 0.014

Efficacy of retinal delivery of AMD 101 by eye drop formulation >100 times of dexamethasone retinal delivery.

The following Table A shows that AMD 101 is excellent in terms of the efficacy of retinal delivery of AMD 101, in comparison to other drugs.

TABLE A

| Compound (brand name) | Drug amount | Drug amount | |
|---|---|---|---|
| MOA for indication or usage | administered | detected in retina | Reference |
| Betoptic 0.5% (MW: 307) | 0.5% | About 300 ng/1 g of | Osborne et al. |
| Beta 1 receptor blocker | 4 drops | retinal tissues | (1999) |
| for glaucoma | (120-200 μL, | (Rabbit) | |
| | 600-1,000 μg) | | |
| Betoptic 0.5% (MW: 307) | 0.5% | About 480 ng/1 g of | Osborne et al. |
| Beta 1 receptor blocker | 8 drops | retinal tissues | (1999) |
| for glaucoma | (240-400 μL, | (Rabbit) | |
| | 1200-2000 μg) | | |
| Dexamethasone (MW: 392) | 0.5% | About 33 ng/1 g of | Sigurdsson et al. |
| Glucocorticoid for | 50 μL, 250 μg | retinal tissues | (2007) |
| inflammation | | (Rabbit) | |
| AMD101 | 0.25%, 3 drops | About 1,288 ng/1 g of | Present study |
| | (15 μL, 37.5 μg) | retinal tissues | |
| | | (Rat) | |
| AMD101 | 0.25%, 10 drops | About 2,939 ng/1 g of | Present study |
| | (50 μL, 125 μg) | retinal tissues | |
| | | (Rat) | |

### Example 4

The present example provides the experimental procedures and results of PK studies showing that rifampicin is delivered to the retina by subcutaneous (SC) injection. The detected amount of rifampicin delivered by SC was equivalent to the detected amount of rifampicin delivered by topical eye drops.

Six male Sprague-Dawley rats (250 to 300 g) were used to measure the retina exposure level of rifampicin after subcutaneous injection (20 mg/Kg). A formulation, which is shown as Example 6A in Table 2, was used, and rifampicin was administered to the rats by SC injection in the present study. At time points of 1 hour, 3 hours, and 7 hours after the SC injection, the retinal tissues were excised from the rats under a dissection microscope. In addition, “non-treated” negative controls were used, and the retina was excised from two rats without performing any treatments on the rats. The retina was placed in a 1.5-ml microcentrifuge tube (1 retina/tube), and was fully washed with DPBS. After completion of the washing procedures, the retinal tissues in the microcentrifuge tube were frozen in dry ice, and were then preserved for quantification by LC/MS analysis. Table 5 shows quantification results of rifampicin, which was extracted from the retinal tissues. The amount of rifampicin detected in the retina, which was delivered by SC, was equivalent to the amount of rifampicin delivered by topical eye drops (see Table 3).

TABLE 5
Amount of rifampicin delivered to retina by subcutaneous injection (20 mg/Kg)

| | Non-treated | 1 hr | 3 hr | 7 hr |
|---|---|---|---|---|
| Rifampicin extracted from retina | BDL | 2,655 | 3,864 | 2,516 |
| (Average of n = 4, | | | | |
| ng/1 g of tissues) | | | | |
| Standard deviation | N/A | 348 | 402 | 447 |

BDL: Below detection limit

### Example 5

The present example provides the experimental procedures and results of PK studies using a 0.25% rifampicin eye drop formulation.

Six male Sprague-Dawley rats (250 to 300 g) were used to measure the retina exposure level of rifampicin after application of the topical eye drops. An eye drop formulation (15 μL), which is shown as Example 1A in Table 1, was used, and the compound was administered to a single eye of each rat. The above-described formulation (15 μL) contained 37.5 lag of rifampicin. At time points of 1 hour, 3 hours, and 7 hours after the application of the eye drops, the retinal tissues were excised from the rats under a dissection microscope. In addition, “non-treated” negative controls were used, and the retina was excised from one rat without performing any treatments on the rats. The retina was placed in a 1.5-ml microcentrifuge tube (1 retina/tube), and was fully washed with DPBS. After completion of the washing procedures, the retinal tissues in the microcentrifuge tube were frozen in dry ice, and were then preserved for quantification by LC/MS analysis. Table 6 shows quantification of rifampicin which was extracted from the retinal tissues. Rifampicin was delivered to the retina by the eye drop formulation (0.25% rifampicin).

TABLE 6
Amount of rifampicin delivered to retina by application
of topical eye drops (0.25% rifampicin)

| | Non-treated | 1 hr | 3 hr | 7 hr |
|---|---|---|---|---|
| Rifampicin extracted from retina | BDL | 1,576 | 1,151 | 104 |
| (Average of n = 4, | | | | |
| ng/1 g of tissues) | | | | |
| Standard deviation | N/A | 868 | 538 | 16 |

BDL: Below detection limit
Tmax: about1 hr,
T½: about 3-4 hr

### Example 6

The present example provides the experimental procedures and results of dose response studies using 0.25% and 0.5% rifampicin eye drop formulations.

Four male Sprague-Dawley rats (250 to 300 g) were used to measure the retina exposure level of rifampicin after the application of topical eye drops. The eye drop formulations (15 μL each), which are shown as Examples 1A and 6A listed in Table 1 and Table 2, were used, and the compounds were each applied to a single eye of each rat. The above-described formulations (15 μL each) contained 37.5 ng and 75 ng of rifampicin, respectively. One hour after the application of the eye drops, the retinal tissues were excised from the rats under a dissection microscope. In addition, “non-treated” negative controls were used, and the retina was excised from one rat without performing any treatments on the rats. The retina was placed in a 1.5-ml microcentrifuge tube (1 retina/tube), and was fully washed with DPBS. After completion of the washing procedures, the retinal tissues in the microcentrifuge tube were frozen in dry ice, and were then preserved for quantification by LC/MS analysis. Table 7 shows quantification of rifampicin, which was extracted from the retinal tissues. Rifampicin was delivered to the retina by application of the two eye drop formulations (0.25% and 0.5 rifampicin) in a dose dependent manner.

TABLE 7
Amount of rifampicin delivered to retina by application
of topical eye drops (0.25% and 0.5% rifampicin)

| | | 0.25% rifampicin | 0.5% rifampicin |
|---|---|---|---|
| | Non-treated | 15 μL | 15 μL |
| Rifampicin extracted from retina | BDL | 1,576 | 6,068 |
| (Average of n = 4, | | | |
| ng/1 g of tissues) | | | |
| Standard deviation | N/A | 868 | 2,634 |

BDL: Below detection limit

### Example 7

The present example provides the experimental procedures of a preclinical efficacy test performed on oxygen-induced retinopathy rat models, using a 0.25% rifampicin eye drop formulation.

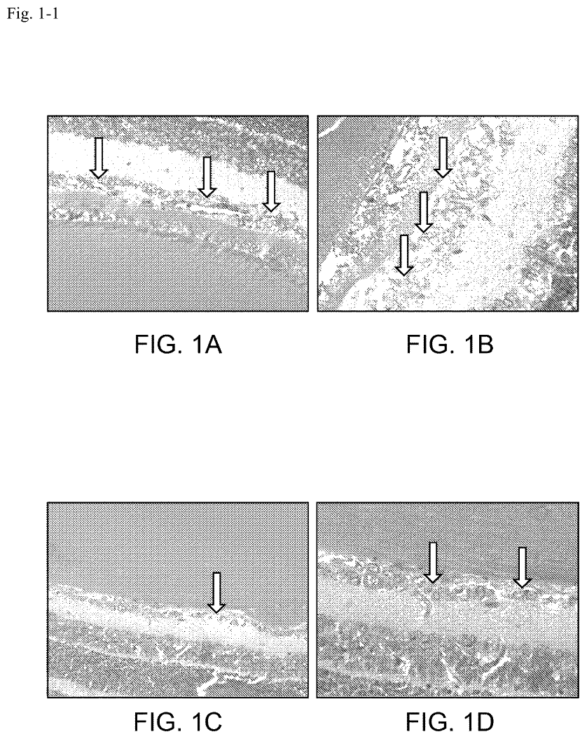

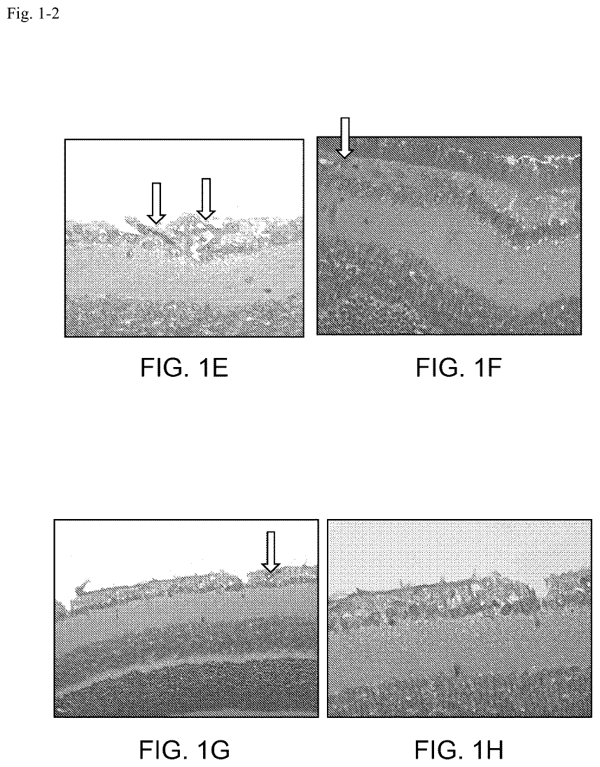

Oxygen-induced retinopathy rat models were produced according to the protocols of Yanni et al. (2010) and Dorfmann et al. (2008). Sprague-Dawley rat babies (and their nursing mothers) were exposed to a cycling oxygen environment (80% and 21%, about one day for each) for 15 days after starting from the day of birth (Day 0). On Day 15 (P15), the animals were moved to room air. Six babies, seven babies, and six babies were assigned to administration groups, namely, a control group of only vehicle, an AMD 101 (rifampicin) eye drop formulation administration group, and an SC injection administration group, respectively. An eye drop formulation shown as Example 1 A in Table 1, or a vehicle only was administered to the eyes of the baby rats every day, for 5 days between P15 and P19, in the morning, about the noon, and in the evening. A formulation shown as Example 6A in Table 2 was used, and AMD 101 (rifampicin) was administered to the baby rats by SC injection at a dose of 20 mg/Kg once a day from P15 to P19. On P20, all of the animals were euthanized, and the retina was visualized as histology sections. In those histology sections, capillaries in the retinal tissues in 3 eye balls (AMD 101 topical application, AMD 101 SC injection administration group, and non-induction of retinopathy) or 5 eye balls (control administration group of a vehicle only) were counted, so that neovascularization was quantified. These eye balls were selected from different animals in the above-described administration groups. Representative images of the histology sections are shown (see FIGS. 1A to 1H). The retina treated with the control of only vehicle showed an increased number of small new capillaries on the retinal surface (see FIGS. 1A and 1B). The retina treated with the AMD 101 topical eye drop formulation showed a small proliferation focus of new capillaries on the retinal surface, but the number of new capillaries was fewer than that in the control group (see FIGS. 1C and 1D). The retina treated with the AMD 101 SC injection showed a small proliferation focus of new capillaries on the retinal surface, but the number of new capillaries is fewer than that in the control group (see FIGS. 1E and 1F). The retina, to which retinopathy was not induced, showed a few small vessel cross sections, but did not show any new capillaries (see FIGS. 1G and 1H). Table 8 shows quantification of capillaries detected in the histology sections.

TABLE 8
Neovascularization in retina of oxygen-induced retinopathy rat models that
was quantified by detection of capillaries in histological sections

| | Non-induction of | Vehicle only | 0.25% AMD101 | 0.5% AMD101 |
|---|---|---|---|---|
| | retinopathy | control | topical eye drop | SC injection |
| | (n = 3) | (n = 5) | (n = 3) | (n = 3) |
| Average numbers of | 3.33 | 16.3 | 7.78 | 9.28 |
| capillaries in high power | | | | |
| field per eye | | | | |
| Standard deviation | 1.109721353 | 1.338468851 | 1.348295678 | 0.895463216 |

Vehicle only vs. 0.25% AMD101 eye drop: P value = 0.0058
Vehicle only vs. 0.5% AMD101 SC: P value = 0.0102

### Example 8

Delivery of ophthalmic formulation comprising rifamycin compounds via a device such as a contact lens.

An ophthalmic formulation comprising an active ingredient, namely, rifampicin, rifabutin, rifapentine, rifalazil or rifaximin, and a pharmaceutically acceptable carrier, and optionally, additives as disclosed herein, is incorporated into an ocular solution used to immerse or wash contact lenses. A contact lens consisting of hydrophilic gel was optionally dried at ambient temperature, and was then immersed in a solution of a soaking agent or a swelling agent, containing an effective amount of the ophthalmic formulation, and was thus washed, or was immersed therein.

### Example 9

### Relationship Between pH and Nonionic Surfactant Concentration for Dissolving Rifamycin Compound

In the present example, the pH value was set to be pH 2 to 12, and the concentration of a nonionic surfactant was set to be 0.001% by weight to 50% by weight. As buffer solutions, a boric acid buffer solution, and a phosphoric acid buffer solution and a citric acid buffer solution were used. Conditions, under which various concentrations of rifampicin are dissolved in the presence of 0.01% benzalkonium chloride (used as both a preservative agent and an ionic surfactant), were studied.

The experimental method is as follows.

A 50 mM phosphoric acid buffer solution, a 50 mM citric acid buffer solution, or a 25 mM boric acid-borax buffer solution (all of which were final concentrations) was prepared, and thereafter, about 0.9% sodium chloride, about 0.1% EDTA, 0.01% benzalkonium chloride (all of which were final concentrations), and various concentrations of nonionic surfactants (Tween 80 or Polyoxyethylene (7) alkyl (sec-C11-15) ethers) were mixed into the solution. To the thus obtained solution, rifampicin (0.0001% by weight to 10% by weight) was added, and the mixture was then blended at 25° C. using a magnetic stirrer. In the case of using a citric acid buffer solution, the pH value of the mixed solution was adjusted using hydrochloric acid under conditions of pH 2.6 or less. In the case of using boric acid, the pH value was adjusted using hydrochloric acid under conditions of pH 5.7 or less. In addition, in the case of using a phosphoric acid buffer solution, the pH value was adjusted using sodium hydroxide under conditions of pH 7.3 or more, and in the case of using boric acid, the pH value was adjusted using sodium hydroxide under conditions of pH 8.6 or more. The thus prepared solutions were continuously blended for about 2 hours, and the pH value thereof was measured at 25° C.

In the present example, the solution, in which precipitation did not occur even though half a day or whole day and night had passed after initiation of the dissolution test and the dissolved state was maintained, was determined to be “dissolved.”

Based on a combination of the pH value and the nonionic surfactant concentration (% by weight) adopted to study conditions, and a coordinate regarding dissolution of rifampicin and a coordinate regarding non-dissolution of rifampicin, in which the X-axis indicates the pH and Y-axis indicates the surfactant concentration, the pH and the nonionic surfactant concentration serving as boundaries are as follows (Tables 9 to 12). The following pH values and concentrations indicate conditions under which rifampicin was dissolved.

TABLE 9
Case of using phosphoric acid buffer solution or citric acid
buffer solution (rifampicin concentration: 0.1% to 10%)

| 0.1% | | 0.25% | | 0.5% | | 1% | | 2% | | 5% | | 10% | |
|---|---|---|---|---|---|---|---|---|---|---|---|---|---|
| pH | % | pH | % | pH | % | pH | % | pH | % | pH | % | pH | % |
| (X) | (Y) | (X) | (Y) | (X) | (Y) | (X) | (Y) | (X) | (Y) | (X) | (Y) | (X) | (Y) |
| 2.0 | 0 | 2.7 | 0.5 | 2.0 | 0 | 2.0 | 0 | 2.2 | 0 | 2.1 | 0 | 1.9 | 0 |
| 2.7 | 0.3 | 4.5 | 1.0 | 2.2 | 0 | 2.5 | 0 | 2.7 | 0.1 | 2.8 | 0.2 | 2.6 | 3 |
| 3.1 | 0.5 | 5.2 | 3 | 3.3 | 1 | 2.9 | 1.5 | 4.1 | 6 | 3.1 | 5 | 2.7 | 5 |
| 3.7 | 0.5 | 5.5 | 3.5 | 4.1 | 2 | 4.0 | 3 | 4.5 | 20 | 3.8 | 12 | 4.1 | 50 |
| 4.5 | 0.4 | 5.9 | 3.5 | 4.3 | 3 | 4.5 | 6 | 5.3 | 40 | 4.2 | 20 | 7.6 | 50 |
| 4.7 | 0.3 | 6.3 | 3 | 4.5 | 5 | 4.6 | 10 | 5.6 | 45 | 4.3 | 35 | 8.8 | 0.001 |
| 5.5 | 0.2 | 6.6 | 2 | 5.3 | 8 | 5.0 | 14 | 5.9 | 45 | 4.5 | 50 | 8.9 | 0 |
| 5.9 | 0.2 | 7.3 | 1 | 5.7 | 9.5 | 5.5 | 19 | 6.2 | 40 | 7.2 | 50 | 11.5 | 0 |
| 6.8 | 0.3 | 7.9 | 0.4 | 6.0 | 8 | 5.9 | 19 | 7.0 | 20 | 7.5 | 30 | | |
| 7.3 | 0.5 | 8.6 | 0.3 | 6.7 | 5 | 6.4 | 14 | 7.3 | 13 | 8.6 | 0.4 | | |
| 8.3 | 0.7 | 10.5 | 0.3 | 7.1 | 3 | 6.8 | 10 | 7.8 | 7 | 8.7 | 0.001 | | |
| 8.9 | 0.7 | 12.2 | 0.4 | 8.2 | 0.3 | 7.3 | 6 | 8.4 | 0.8 | 10.6 | 0.001 | | |
| 9.5 | 0.6 | | | 9.0 | 0.2 | 8.2 | 2 | 8.7 | 0.1 | 11.3 | 0.01 | | |
| 12.0 | 0.6 | | | 11.0 | 0.2 | 8.3 | 0.5 | 11.8 | 0.1 | 11.6 | 0.03 | | |
| | | | | | | 8.7 | 0.2 | | | | | | |
| | | | | | | 11.9 | 0.2 | | | | | | |

TABLE 10
Case of using phosphoric acid buffer solution or citric acid
buffer solution (rifampicin concentration: 0.0001% to 0.05%)

| 0.0001% | | 0.0005% | | 0.001% | | 0.01% | | 0.05% | |
|---|---|---|---|---|---|---|---|---|---|
| pH | % | pH | % | pH | % | pH | % | pH | % |
| (X) | (Y) | (X) | (Y) | (X) | (Y) | (X) | (Y) | (X) | (Y) |
| 7.0 | 0 | 2.7 | 0 | 2.8 | 0 | 2.0 | 0.1 | 2.0 | 0.3 |
| 7.8 | * | 4.7 | 0 | 4.8 | 0 | 3.8 | 0.3 | 2.9 | 0.4 |
| 8.0 | * | 6.9 | 0 | 6.3 | 0 | 5.5 | 0.3 | 4.9 | 0.4 |
| 9.0 | * | 8.0 | * | 7.0 | 0.001 | 5.6 | 0 | 5.9 | 0.2 |
| 10.7 | 0 | 9.0 | * | 8.8 | 0.001 | 6.0 | 0.01 | 6.9 | 0.4 |
| | | 10.7 | * | 10.6 | 0.001 | 6.5 | 0.2 | 9.0 | 0.7 |
| | | 12.2 | 0 | 12.0 | 0 | 7.9 | 0.5 | 10.4 | 0.7 |
| | | | | | | 10.6 | 0.5 | 11.9 | 0.7 |
| | | | | | | 12.1 | 0.6 | | |

(* indicates that a precipitate was generated under conditions of not adding nonionic surfactants.)

TABLE 11
Case of using boric acid buffer solution (rifampicin concentration: 0.1% to 10%)

| 0.1% | | 0.25% | | 0.5% | | 1% | | 2% | | 5% | | 10% | |
|---|---|---|---|---|---|---|---|---|---|---|---|---|---|
| pH | % | pH | % | pH | % | pH | % | pH | % | pH | % | pH | % |
| (X) | (Y) | (X) | (Y) | (X) | (Y) | (X) | (Y) | (X) | (Y) | (X) | (Y) | (X) | (Y) |
| 2.1 | 0.4 | 2.3 | 0.8 | 2.0 | 1.3 | 2.4 | 4.6 | 2.4 | 6 | 2.1 | 24 | 2.1 | 40 |
| 3.2 | 0.3 | 3.4 | 0.7 | 2.4 | 1.1 | 3.0 | 2.6 | 3.0 | 7 | 2.7 | 12 | 2.6 | 30 |
| 4.9 | 0.2 | 4.3 | 0.6 | 2.7 | 1.1 | 4.2 | 3.4 | 3.7 | 7 | 3.6 | 12 | 2.4 | 20 |
| 6.4 | 0.2 | 7.1 | 0.6 | 3.4 | 1.3 | 5.1 | 3.8 | 4.6 | 9 | 4.6 | 26 | 3.6 | 20 |
| 6.7 | 0.3 | 7.6 | 0.5 | 6.3 | 1.3 | 6.3 | 3.8 | 5.5 | 9 | 5.2 | 38 | 3.7 | 30 |
| 7.5 | 0.3 | 8.2 | 0.3 | 6.4 | 1.2 | 6.9 | 3.4 | 6.1 | 8 | 5.6 | 46 | 4.2 | 50 |
| 8.3 | 0.3 | 8.7 | 0.2 | 6.6 | 1.0 | 7.1 | 2.6 | 7.0 | 7 | 6.0 | 30 | 7.2 | 50 |
| 9.1 | 0.3 | 12.0 | 0.2 | 7.2 | 0.7 | 7.4 | 1.8 | 7.9 | 3 | 7.5 | 15 | 8.6 | 0 |
| 12.0 | 0.3 | | | 7.9 | 0.3 | 7.8 | 0.9 | 8.2 | 0.6 | 8.2 | 5 | 9.6 | 0 |
| | | | | 8.9 | 0.2 | 8.5 | 0.2 | 8.8 | 0.1 | 8.5 | 0 | 11.1 | 0 |
| | | | | 10.9 | 0.2 | 9.7 | 0.1 | 11.7 | 0.1 | 9.9 | 0 | | |
| | | | | | | 10.0 | 0.1 | | | 11.7 | 0 | | |
| | | | | | | 11.4 | 0.2 | | | | | | |

TABLE 12
Case of using boric acid buffer solution (rifampicin
concentration: 0.001% to 0.05%)

| 0.0001% | | 0.0005% | | 0.001% | | 0.01% | | 0.05% | |
|---|---|---|---|---|---|---|---|---|---|
| pH | % | pH | % | pH | % | pH | % | pH | % |
| (X) | (Y) | (X) | (Y) | (X) | (Y) | (X) | (Y) | (X) | (Y) |
| 7.1 | 0 | 2.1 | 0 | 2.0 | 0 | 2.0 | 0.05 | 2.1 | 0.2 |
| 8.8 | 0 | 3.9 | 0 | 3.9 | 0 | 3.9 | 0.01 | 4.2 | 0.2 |
| 9.9 | * | 5.9 | 0 | 6.0 | 0 | 6.0 | 0.001 | 5.9 | 0.2 |
| 10.8 | 0 | 7.1 | * | 7.5 | 0.001 | 7.0 | 0.1 | 6.9 | 0.3 |
| | | 8.0 | * | 8.8 | 0.001 | 8.0 | 0.3 | 7.9 | 0.4 |
| | | 10.7 | * | 9.8 | 0.001 | 12.0 | 0.3 | 10.4 | 0.4 |
| | | 12.1 | 0 | 10.9 | 0 | | | 12.0 | 0.4 |
| | | | | 12.1 | 0 | | | | |

(* indicates that a precipitate was generated under conditions of not adding nonionic surfactants.)

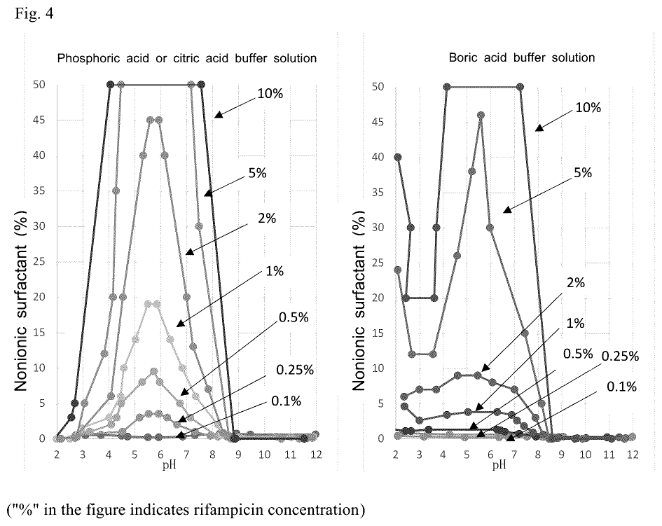

The results obtained by showing the boundary between the soluble region and the insoluble region in the form of a graph, based on the above-described results, are shown in FIG. 3 and FIG. 4 (wherein the line in the graph indicates a soluble region). Herein, a region that can be determined to be dissolved without plotting can be explained as follows. That is, a hardly-soluble compound such as rifampicin can be solubilized by being mixed with a nonionic surfactant to form a micelle. After the formation of a micelle, even if a nonionic surfactant is further added thereto additionally, the compound has already been solubilized as a result of the formation of a micelle, and thus, an insoluble precipitate is not generated by inhibition of the soluble state. Even in the actual experimental results, the soluble state did not change, even though a nonionic surfactant was added in a concentration higher than the boundary.

FIG. 4 shows a solubility curve, in which the longitudinal axis indicates the concentration (%) of a nonionic surfactant, the horizontal axis indicates pH, the above-described coordinate is divided into the case of using a phosphoric acid/citric acid buffer solution and the case of using a boric acid buffer solution, followed by plotting (see FIG. 4). As described in FIG. 2, FIG. 4 shows that the compound was soluble under the composition conditions on the curve of each solubility curve and the upper side thereof, whereas the compound was insoluble under the composition conditions on the lower side thereof. The solubility curve of a phosphoric acid/citric acid buffer solution had almost the same results, even using buffer solutions other than a boric acid buffer solution (e.g. an acetic acid buffer solution or a Tris-HCl buffer solution). Moreover, almost the same results were obtained even using polyoxyethylene hydrogenated castor oil or polyoxy castor oil as a nonionic surfactant.

With regard to the relationship between the area under the curve of the solubility curve shown in FIG. 4 and each rifampicin concentration, when the longitudinal axis was set to be the area under the curve AUC (the product of the pH with the nonionic surfactant concentration (%)), the horizontal axis was set to be the rifampicin concentration (%), followed by plotting, an approximate relationship was found. FIG. 5 shows that the condition is divided into the case of using a phosphoric acid/citric acid buffer solution and the case of using a boric acid buffer solution, and that the relationship of the two cases is approximated to the curve.

In FIG. 5, the relationship between the rifampicin concentration (x) and the AUC (y_{AUC}) in the case of using a phosphoric acid/citric acid buffer solution is approximated by the equation: y_{AUC}=6.1548x²+13.293x+0.6968, whereas the relationship between the rifampicin concentration (x) and the AUC (y_{AUC}) in the case of using a boric acid buffer solution is approximated by the equation: y_{AUC}=0.6733x²+7.5348x−0.1957. Since AUC is represented by the product of the pH with the nonionic surfactant concentration (%), it is demonstrated that, with a change in the rifampicin concentration, the solubilized dose is also changed in the range of the above-described relational expression.

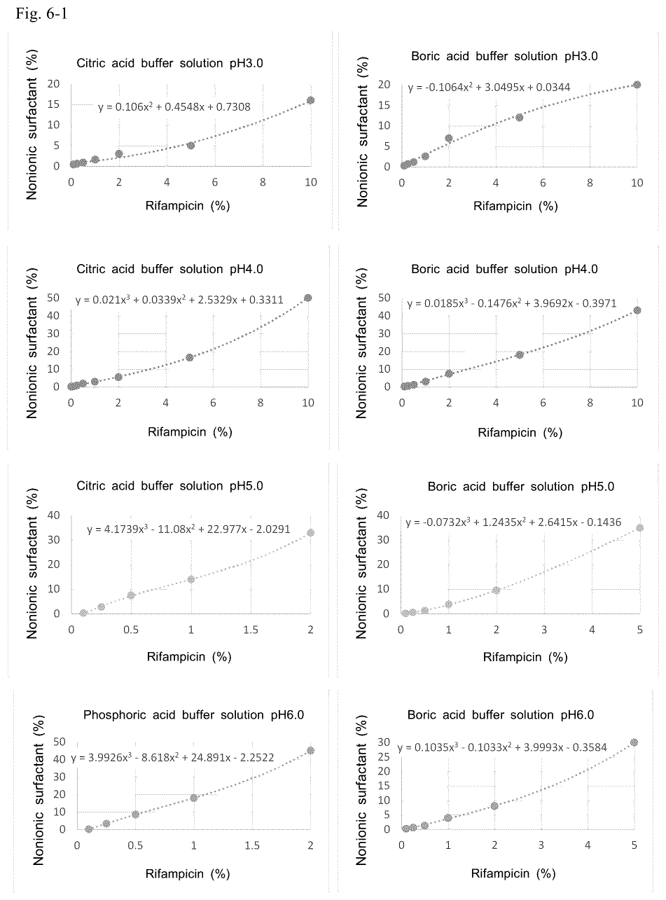

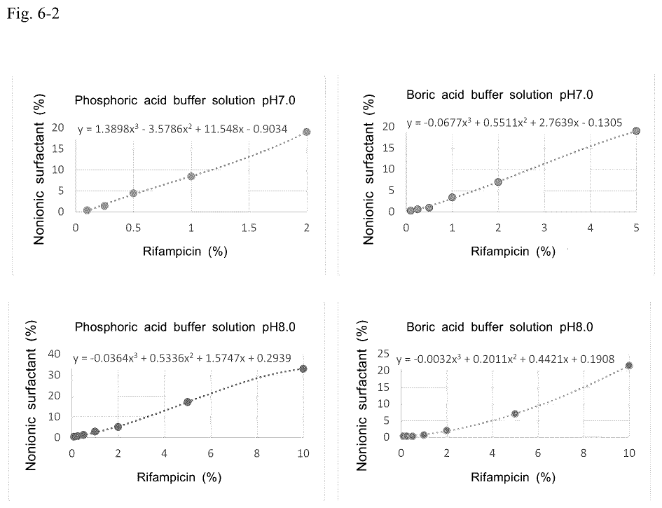

In order to show the relationship between each rifampicin concentration (%) and the nonionic surfactant concentration (%) required for solubilization, the nonionic surfactant concentration (%) required for solubilization for every pH 1 from pH 3.0 to pH 8.0 in the solubility curve shown in FIG. 4 and each rifampicin concentration (%) were plotted. As a result, the nonionic surfactant concentration (%) required for the solubilization for any given rifampicin concentration was precited (see FIG. 6).

In FIG. 6, the relationship between the rifampicin concentration (x) and the nonionic surfactant (y_{Nis}) at each pH value in the case of using a phosphoric acid/citric acid buffer solution is represented by the following equations. In this case, the composition further comprises an ionic surfactant in a range of more than 0.00005% and 0.01% or less.

y_{Nis}=0.106x²+0.4548x+0.7308(pH3.0)

y_{Nis}=0.0021x³+0.0339x²+2.5329x+0.3311(pH4.0)

y_{Nis}=4.1739x³−11.08x²+22.977x−2.0291(pH5.0)

y_{Nis}=3.9926x³−8.618x²+24.891x−2.2522(pH6.0)

y_{Nis}=1.3898x³−3.5786x²+11.548x−0.9034(pH7.0)

y_{Nis}=−0.0364x³+0.5336x²+1.5747x+0.2939(pH8.0)

In addition, in FIG. 6, the relationship between the rifampicin concentration (x) and the nonionic surfactant (y_{Nis}) at each pH value in the case of using a boric acid buffer solution is represented by the following equations. In this case, the composition further comprises an ionic surfactant in a range of more than 0.00005% and 0.01% or less.

y_{Nis}=−0.1064x²+3.0495x+0.0344(pH3.0)

y_{Nis}=0.0185x³−0.1476x²+3.9692x−0.3971(pH4.0)

y_{Nis}=−0.0732x³+1.2435x²+2.6415x−0.1436(pH5.0)

y_{Nis}=0.1035x³−0.1033x²+3.9993x−0.3584(pH6.0)

y_{Nis}=−0.0677x³+0.5511x²+2.7639x−0.1305(pH7.0)

y_{Nis}=−0.0032x³+0.2011x²+0.4421x+0.1908(pH8.0)

As an example, in the case of a 3% rifampicin solution using a boric acid buffer solution, if the above-described relational expression is used, the composition is solubilized by addition of about 8.2% of a nonionic surfactant at pH 3.0, whereas the composition is solubilized by addition of about 10.7% of a nonionic surfactant at pH 4.0. Accordingly, in the pH range of pH 3.0 to pH 4.0, the composition becomes solubilized by addition of a nonionic surfactant in the range of about 8.2% to about 10.7%. Thus, the range of the additive amount of a nonionic surfactant, which is necessary for solubilization at any given pH value, is predicted.

### Example 10

### Relationship Between pH and Nonionic Surfactant Concentration for Dissolution of Rifamycin Compound

In the present example, the pH value was set to be pH 2.000 to 12.000, and the concentration of a nonionic surfactant was set to be 0.01 to 10.0. As buffer solutions, a boric acid buffer solution, and a phosphoric acid buffer solution and a citric acid buffer solution were used. Conditions, under which various concentrations of rifampicin are dissolved, were studied. Besides, the concentration of benzalkonium chloride (used as both a preservative agent and an ionic surfactant) was adjusted in the range of 0%, or more than 0% and 0.00005% or less, as a final concentration.

The experimental method is as follows.

A 50 mM phosphoric acid buffer solution, a 50 mM citric acid buffer solution, or a 25 mM boric acid-borax buffer solution (all of which were final concentrations) was prepared, and thereafter, about 0.9% sodium chloride, about 0.1% EDTA, (all of which were final concentrations), and various concentrations of nonionic surfactants (Polyoxyethylene (7) alkyl (sec-C11-15) ethers) were mixed into the solution. To the thus obtained solution, rifampicin (0.0001% by weight to 10% by weight) was added, and the mixture was then blended at 25° C. using a magnetic stirrer. In the case of using a citric acid buffer solution, the pH value of the mixed solution was adjusted using hydrochloric acid under conditions of pH 2.6 or less. In the case of using boric acid, the pH value was adjusted using hydrochloric acid under conditions of pH 5.7 or less. In addition, in the case of using a phosphoric acid buffer solution, the pH value was adjusted using sodium hydroxide under conditions of pH 7.3 or more, and in the case of using boric acid, the pH value was adjusted using sodium hydroxide under conditions of pH 8.6 or more. The thus prepared solutions were continuously blended for about 2 hours, and the pH value thereof was measured at 25° C.

In the present example, the solution, in which precipitation did not occur even though half a day or whole day and night had passed after initiation of the dissolution test and the dissolved state was maintained, was determined to be “dissolved.”

Based on a combination of the pH value and the nonionic surfactant concentration (% by weight) adopted to study conditions, and a coordinate regarding dissolution of rifampicin and a coordinate regarding non-dissolution of rifampicin, in which the X-axis indicates the pH and Y-axis indicates the surfactant concentration, the pH and the nonionic surfactant concentration serving as boundaries (the concentration as a condition for dissolution of rifampicin) are as follows (Tables 13 to 16).

TABLE 13
Case of using phosphoric acid buffer solution or citric acid
buffer solution (rifampicin concentration: 0.1% to 10%)

| 0.1% | | 0.5% | | 1% | | 2% | | 5% | | 10% | |
|---|---|---|---|---|---|---|---|---|---|---|---|
| pH | % | pH | % | pH | % | pH | % | pH | % | pH | % |
| (X) | (Y) | (X) | (Y) | (X) | (Y) | (X) | (Y) | (X) | (Y) | (X) | (Y) |
| 2.0 | 0 | 2.1 | 0 | 2.5 | 0 | 2.1 | 0 | 8.7 | 0 | 8.9 | 0 |
| 2.9 | 0.5 | 3.3 | 1.2 | 3.0 | 1.5 | 3.2 | 3 | 10.4 | 0 | 9.8 | 0 |
| 3.8 | 0.4 | 4.2 | 1.8 | 4.0 | 3 | 4.0 | 6 | 11.6 | 0 | 11.6 | 0 |
| 4.8 | 0.4 | 4.9 | 7.5 | 5.0 | 18 | 4.8 | 30 | | | | |
| 6.0 | 1.3 | 5.9 | 9.5 | 6.0 | 20 | 5.8 | 45 | | | | |
| 6.8 | 0.9 | 6.9 | 5 | 7.0 | 10 | 7.8 | 7 | | | | |
| 7.8 | 0.3 | 9.0 | 0 | 7.8 | 5 | 8.8 | 0 | | | | |
| 8.9 | 0 | 10.0 | 0 | 8.6 | 0.1 | 9.5 | 0 | | | | |
| 10.5 | 0 | 10.9 | 0 | 8.7 | 0 | 10.9 | 0 | | | | |
| 12.0 | 0 | | | 9.9 | 0 | 11.8 | 0 | | | | |
| | | | | 11.8 | 0 | | | | | | |

TABLE 14
Case of using phosphoric acid buffer solution or citric acid
buffer solution (rifampicin concentration: 0.002% to 0.05%)

| | | | |
|---|---|---|---|
| 0.002% | 0.005% | 0.01% | 0.05% |

| pH | % | pH | % | pH | % | pH | % |
|---|---|---|---|---|---|---|---|
| (X) | (Y) | (X) | (Y) | (X) | (Y) | (X) | (Y) |
| 2.7 | 0 | 2.7 | 0.01 | 2.2 | 0.2 | 2.0 | 0.3 |
| 4.8 | 0 | 3.8 | 0 | 2.9 | 0.2 | 2.9 | 0.4 |
| 6.9 | 0 | 5.5 | 0 | 3.9 | 0.02 | 3.8 | 0.4 |
| 8.6 | 0 | 6.8 | 0 | 4.8 | 0 | 4.9 | 0.3 |
| 10.4 | 0 | 8.3 | 0 | 5.8 | 0 | 5.9 | 0.5 |
| 11.9 | 0 | 10.4 | 0 | 6.8 | 0 | 6.8 | 0 |
| | | 12.0 | 0 | 7.9 | 0 | 9.0 | 0 |
| | | | | 10.2 | 0 | 10.6 | 0 |
| | | | | 11.8 | 0 | 12.1 | 0 |

TABLE 15
Case of using boric acid buffer solution
(rifampicin concentration: 0.1% to 10%)

| 0.1% | | 0.5% | | 1% | | 2% | | 5% | | 10% | |
|---|---|---|---|---|---|---|---|---|---|---|---|
| pH | % | pH | % | pH | % | pH | % | pH | % | pH | % |
| (X) | (Y) | (X) | (Y) | (X) | (Y) | (X) | (Y) | (X) | (Y) | (X) | (Y) |
| 2.1 | 0.4 | 3.1 | 1.3 | 2.3 | 3.0 | 2.4 | 6 | 2.1 | 14 | 8.6 | 0 |
| 3.2 | 0.3 | 4.1 | 1.3 | 3.2 | 2.5 | 3.0 | 7 | 2.6 | 14 | 9.6 | 0 |
| 4.1 | 0.2 | 6.5 | 1.3 | 4.3 | 3.0 | 4.2 | 6 | 3.6 | 12 | 11.4 | 0 |
| 5.0 | 0.1 | 7.0 | 1.0 | 5.1 | 4.5 | 5.9 | 10 | 4.5 | 20 | | |
| 5.8 | 0.1 | 7.4 | 0.8 | 6.2 | 3.8 | 7.0 | 6 | 5.5 | 46 | | |
| 6.8 | 0.1 | 7.5 | 0.5 | 6.9 | 3.4 | 7.9 | 2 | 5.9 | 30 | | |
| 7.8 | 0 | 8.0 | 0.1 | 7.8 | 0.9 | 8.8 | 0 | 6.7 | 20 | | |
| 9.1 | 0 | 8.9 | 0 | 8.9 | 0.1 | 9.9 | 0 | 8.2 | 5 | | |
| 10.4 | 0 | 9.7 | 0 | 8.9 | 0 | 11.5 | 0 | 8.5 | 0 | | |
| 12.0 | 0 | 11.6 | 0 | 10.0 | 0 | | | 9.1 | 0 | | |
| | | | | 11.4 | 0 | | | 11.2 | 0 | | |

TABLE 16
Case of using boric acid buffer solution (rifampicin
concentration: 0.002% to 0.05%)

| | | | |
|---|---|---|---|
| 0.002% | 0.005% | 0.01% | 0.05% |

| pH | % | pH | % | pH | % | pH | % |
|---|---|---|---|---|---|---|---|
| (X) | (Y) | (X) | (Y) | (X) | (Y) | (X) | (Y) |
| 2.0 | 0 | 2.0 | 0.01 | 2.0 | 0.1 | 2.0 | 0.3 |
| 3.9 | 0 | 3.0 | 0 | 3.7 | 0 | 3.1 | 0.2 |
| 6.0 | 0 | 4.9 | 0 | 5.7 | 0 | 4.1 | 0.2 |
| 7.9 | 0 | 6.9 | 0 | 7.2 | 0 | 4.8 | 0.1 |
| 9.7 | 0 | 8.7 | 0 | 8.6 | 0 | 6.8 | 0.1 |
| 11.9 | 0 | 10.2 | 0 | 10.0 | 0 | 7.9 | 0 |
| | | 11.9 | 0 | 11.8 | 0 | 10.7 | 0 |
| | | | | | | 12.1 | 0 |

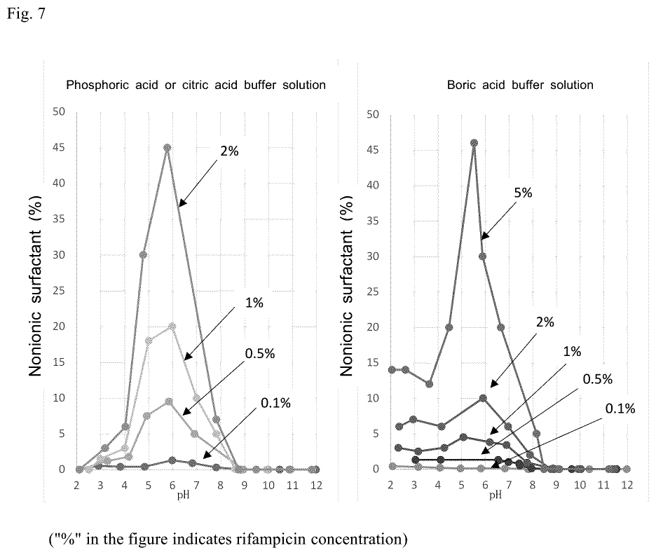

Based on the above-described results, as with the method described in FIG. 4, the soluble region and the insoluble region was shown in a graph, for individual rifampicin concentrations (FIG. 7). The longitudinal axis indicates the concentration (%) of a nonionic surfactant, and the horizontal axis indicates pH. As described in FIG. 2, in the solubility curve shown in FIG. 7, the region on the curve and the upper side thereof constitute a soluble region, and the lower side thereof is an insoluble region. The solubility curve shown in FIG. 7 was obtained by addition of benzalkonium chloride (final concentration: 0%, or more than 0% and 0.00005% or less). On the other hand, the solubility curve shown in FIG. 4 was obtained by addition of benzalkonium chloride (final concentration: 0.01%). The two solubility curves were similar to each other.

Moreover, the above-described results demonstrated that, if the pH is, at least, about pH 8.5 or more, solubilization of rifampicin in a rifampicin concentration of 0.002% by weight to 10% by weight does not need addition of a surfactant. Furthermore, in the pH range of pH 2 to pH 12, solubilization of rifampicin in a rifampicin concentration of 0.002% by weight or less did not need addition of a surfactant. Further, it was demonstrated that when an ionic surfactant is added in a concentration of more than 0.0005% by weight, rifampicin is solubilized by setting the concentration of a nonionic surfactant to be 0% (not added) to 15%, at pH 8.5 or more.

With regard to the relationship between the area under the curve of the solubility curve shown in FIG. 7 and each rifampicin concentration, when the longitudinal axis was set to be the area under the curve AUC (the product of the pH with the nonionic surfactant concentration (%)), the horizontal axis was set to be the rifampicin concentration (%), followed by plotting, an approximate relationship was found. FIG. 8 shows that the condition is divided into the case of using a phosphoric acid/citric acid buffer solution and the case of using a boric acid buffer solution, and that the relationship of the two cases is approximated to the curve.

In FIG. 8, the relationship between the rifampicin concentration (x) and the AUC (y_{AUC}) in the case of using a phosphoric acid/citric acid buffer solution is approximated by the equation: y_{AUC}=1.2623x²+24.21x−0.2533, whereas the relationship between the rifampicin concentration (x) and the AUC (y_{AUC}) in the case of using a boric acid buffer solution is approximated by the equation: y_{AUC}=0.647x²+7.8665x−0.0208. As with FIG. 5, since AUC is represented by the product of the pH with the nonionic surfactant concentration (%), it is demonstrated that, with a change in the rifampicin concentration, the solubilized dose is also changed in the range of the above-described relational expression.

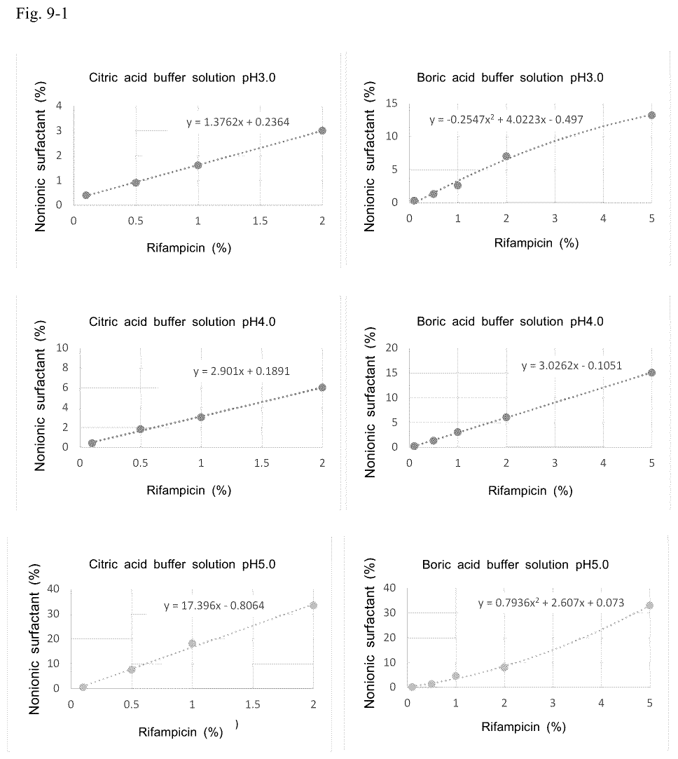

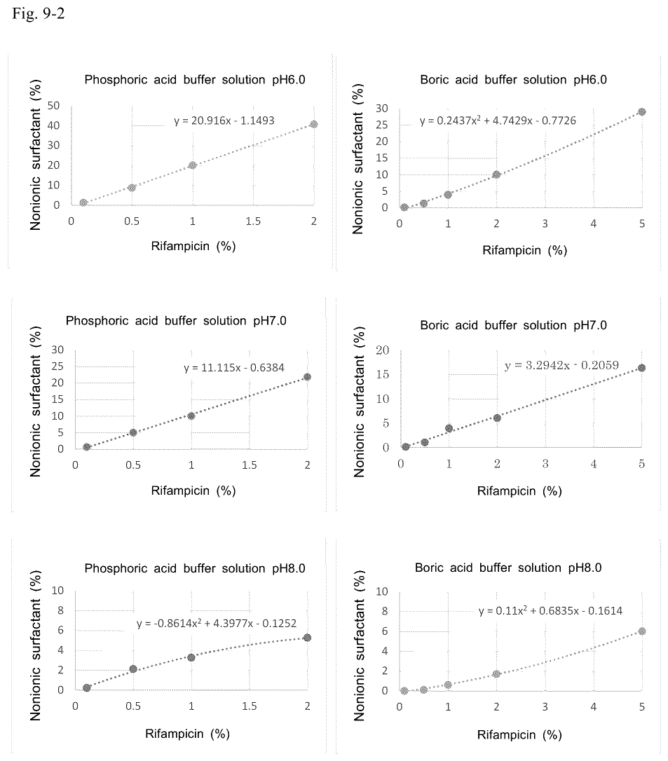

In order to show the relationship between each rifampicin concentration (%) and the nonionic surfactant concentration (%) required for solubilization, the nonionic surfactant concentration (%) required for solubilization for every pH 1 from pH 3.0 to pH 8.0 in the solubility curve shown in FIG. 7 and each rifampicin concentration (%) were plotted. As a result, the nonionic surfactant concentration (%) required for the solubilization for any given rifampicin concentration was precited (see FIG. 9).

In FIG. 9, the relationship between the rifampicin concentration (x) and the nonionic surfactant (y_{Nis}) at each pH value in the case of using a phosphoric acid/citric acid buffer solution is represented by the following equations. In this case, 0.00005% or less of an ionic surfactant is further comprised, or is not comprised.

y_{Nis}=1.3762x+0.2364(pH3.0)

y_{Nis}=2.901x+0.1891(pH4.0)

y_{Nis}=17.396x−0.8064(pH5.0)

y_{Nis}=20.916x−1.1493(pH6.0)

y_{Nis}=11.115x−0.6384(pH7.0)

y_{Nis}=−0.8614x²+4.3977x−0.1255(pH8.0)

In addition, in FIG. 9, the relationship between the rifampicin concentration (x) and the nonionic surfactant (y_{Nis}) in the case of using a boric acid buffer solution is represented by the following equation. In this case, 0.00005% or less of an ionic surfactant is further comprised, or is not comprised.

y_{Nis}=−0.2547x²+4.0223x−0.497(pH3.0)

y_{Nis}=3.0262x−0.1051(pH4.0)

y_{Nis}=0.7936x²+2.607x+0.073(pH5.0)

y_{Nis}=0.2437x²+4.7429x−0.7726(pH6.0)

y_{Nis}=3.2942x−0.2059(pH7.0)

y_{Nis}=0.11x²+0.6835x−0.1614(pH8.0)

By using the above-describe relational equations, the range of additive amount of the nonionic surfactant at any given pH value can be predicted, as with the explanation of FIG. 6.

It will be apparent to those skilled in the art that various modifications and variations can be made on the method and composition of the present invention without departing from the spirit or scope of the present invention. Therefore, it is intended that the present invention includes such modifications and variations of the present invention, as long as those modifications and variations are included in the scope of the appended claims and equivalents thereto.

1. A topical eye drop composition, which comprises an aqueous solution formulation comprising an effective amount of a rifamycin compound selected from the group consisting of rifampicin, rifabutin, rifapentine, and rifaximin, or a pharmaceutically acceptable salt thereof, a buffer solution, and 0.00001% to 50% by weight of a nonionic surfactant, wherein
the pH of the aqueous solution formulation is pH 2 to 12, and
when the concentration (% by weight) of the nonionic surfactant and the concentration (% by weight) of the rifamycin compound satisfy the equation represented by the following formula (2) under any of the following conditions (i) to (iv), the rifamycin compound or a pharmaceutically acceptable salt thereof is stably dissolved in the aqueous solution formulation:
y_{Nis}=ax³+bx²+cx+d (2)
wherein y_{Nis} represents the concentration of the nonionic surfactant, and x represents the concentration of the rifamycin compound,
(i) the aqueous solution formulation further comprises more than 0.00005% of an ionic surfactant, and when a buffer solution other than a boric acid buffer solution is used, the following holds in the formula (2):
(a, b, c, and d) (pH)=
(0, 0.106, 0.4548, 0.7308) (pH 3.0),
(0.0021, 0.0339, 2.5329, 0.3311) (pH 4.0),
(4.1739, −11.08, 22.977, −2.0291) (pH 5.0),
(3.9926, −8.618, 24.891, −2.2522) (pH 6.0),
(1.3898, −3.5786, 11.548, −0.9034) (pH 7.0) or
(−0.0364, 0.5336, 1.5747x, 0.2939) (pH 8.0),
(ii) the aqueous solution formulation further comprises more than 0.00005% of an ionic surfactant, and when a boric acid buffer solution is used, the following holds in the formula (2):
(a, b, c, and d) (pH)=
(0, −0.1064, 3.0495, 0.0344) (pH 3.0),
(0.0185, −0.1476, 3.9692, −0.3971) (pH 4.0),
(−0.0732, 1.2435, 2.6415, −0.1436) (pH 5.0),
(0.1035, −0.1033, 3.9993, −0.3584) (pH 6.0),
(−0.0677, 0.5511, 2.7639, −0.1305) (pH 7.0) or
(−0.0032, 0.2011, 0.4421, 0.1908) (pH 8.0),
(iii) the aqueous solution formulation further comprises 0.00005% or less of an ionic surfactant or does not comprise such an ionic surfactant, and when a buffer solution other than a boric acid buffer solution is used, the following holds in the formula (2):
(a, b, c, and d) (pH)=
(0, 0, 1.3762, 0.2364) (pH 3.0),
(0, 0, 2.901, 0.1891) (pH 4.0),
(0, 0, 17.396, −0.8064) (pH 5.0),
(0, 0, 20.916, −1.1493) (pH 6.0),
(0, 0, 11.115, −0.6384) (pH 7.0) or
(0, −0.8614, 4.3977, −0.1255) (pH 8.0), and
(iv) the aqueous solution formulation further comprises 0.00005% or less of an ionic surfactant or does not comprise such an ionic surfactant, and when a boric acid buffer solution is used, the following holds in the formula (2):
(a, b, c, and d) (pH)=
(0, −0.2547, 4.0223, −0.497) (pH 3.0),
(0, 0, 3.0262, −0.1051) (pH 4.0),
(0, 0.7936, 2.607, 0.073) (pH 5.0),
(0, 0.2437, 4.7429, −0.7726) (pH 6.0),
(0, 0, 3.2942, −0.2059) (pH 7.0) or
(0, 0, 0.11, 0.6835, −0.1614) (pH 8.0).

2. A topical eye drop composition, which comprises an aqueous solution formulation comprising an effective amount of a rifamycin compound selected from the group consisting of rifampicin, rifabutin, rifapentine, and rifaximin, or a pharmaceutically acceptable salt thereof, a buffer solution, and optionally, an ionic surfactant and/or a nonionic surfactant, wherein
when the rifamycin compound, ionic surfactant, nonionic surfactant, and pH have the following relationship A1 or A2, the rifamycin compound or a pharmaceutically acceptable salt thereof is stably dissolved in the aqueous solution formulation:
(A1) the aqueous solution formulation comprises 0% (not comprise) to 0.00005% by weight of an ionic surfactant and does not comprise a nonionic surfactant, and the concentration of the rifamycin compound is 0.002% to 10% by weight, and the pH of the aqueous solution formulation is pH 8.5 or more, or
(A2) the aqueous solution formulation comprises more than 0.0005% by weight of an ionic surfactant, and the concentration of the nonionic surfactant is 0% (not added) to 15% by weight, and the concentration of the rifamycin compound is 0.0001% to 10% by weight, and the pH of the aqueous solution formulation is pH 8.5 or more.

3. The composition according to claim 1 or 2, further comprising a pharmaceutically acceptable carrier.

4. The composition according to claim 3, wherein the pharmaceutically acceptable carrier comprises a tonicity adjusting agent.

5. The composition according to claim 4, wherein the tonicity adjusting agent is saline, dextrose, glycerin, potassium chloride aqueous solution, buffer salts, propylene glycol, or mannitol.

6. The composition according to claim 1, which is used for at least one selected from the group consisting of macular degeneration, diabetic retinopathy, chronic glaucoma, retinal detachment, sickle cell retinopathy, age-related macular degeneration (AMD), retinal ganglion cell injury, rubeosis iritis, inflammatory disease, chronic uveitis, neoplasm, Fuchs' heterochromic iridocyclitis, neovascular glaucoma, corneal neovascularization, choroidal neovascularization, retinal neovascularization, retinal angiomatous proliferation, glaucoma, glaucoma surgery, tissue adhesion, cicatrization, tissue fibrosis, and brain damage.

7. The composition according to claim 1, further comprising one or more of an antibacterial agent, a pH buffer agent, an antioxidant, a preservative agent, or a combination thereof.

8. The composition according to claim 1, wherein the rifamycin compound is rifampicin.

9. The composition according to any one of claim 1, wherein the concentration of the rifamycin compound or a pharmaceutically acceptable salt thereof is 0.1% to 10% by weight.

10. A medicament for treatment of an ocular disease, disorder or condition, wherein the medicament comprises the composition according to claim 1.

11. The medicament according to claim 10, wherein the ocular disease, disorder or condition is at least one selected from the group consisting of macular degeneration, diabetic retinopathy, chronic glaucoma, retinal detachment, sickle cell retinopathy, age-related macular degeneration (AMD), retinal ganglion cell injury, rubeosis iritis, inflammatory disease, chronic uveitis, neoplasm, Fuchs' heterochromic iridocyclitis, neovascular glaucoma, corneal neovascularization, choroidal neovascularization, retinal neovascularization, retinal angiomatous proliferation, glaucoma, glaucoma surgery, tissue adhesion, cicatrization, tissue fibrosis, and brain damage.
